SEQUENCE LISTING (19 sequences)

SEQ ID NO: 1 - protein, 524 aa, Homo sapiens
MPTVEELYRNYGILADATEQVGQHKDAYQVILDGVKGGTKEKRLAAQFIPKFFKHFPELADSAINAQLDLCEDEDVSIRRQAIKELPQFATGENLPRVADILTQLLQTDDSAEFNLVNNALLSIFKMDAKGTLGGLFSQILQGEDIVRERAIKFLSTKLKTLPDEVLTKEVEELILTESKKVLEDVTGEEFVLFMKILSGLKSLQTVSGRQQLVELVAEQADLEQTFNPSDPDCVDRLLQCTRQAVPLFSKNVHSTRFVTYFCEQVLPNLGTLTTPVEGLDIQLEVLKLLAEMSSFCGDMEKLETNLRKLFDKLLEYMPLPPEEAENGENAGNEEPKLQFSYVECLLYSFHQLGRKLPDFLTAKLNAEKLKDFKIRLQYFARGLQVYIRQLRLALQGKTGEALKTEENKIKVVALKITNNINVLIKDLFHIPPSYKSTVTLSWKPVQKVEIGQKRASEDTTSGSPPKKSSAGPKRDARQIYNPPSGKYSSNLGNFNYEQRGAFRGSRGGRGWGTRGNRSRGRLY

SEQ ID NO: 2 - protein, 16 aa, Artificial Sequence
  Synthetic Penetratin alone
RQIKIWFQNRRMKWKK

SEQ ID NO: 3 - protein, 37 aa, Homo sapiens
AKLNAEKLKDFKIRLQYFARGLQVYIRQLRLALQGKT

SEQ ID NO: 4 - protein, 53 aa, Homo sapiens
RQIKIWFQNRRMKWKKAKLNAEKLKDFKIRLQYFARGLQVYIRQLRLALQGKT

SEQ ID NO: 5 - protein, 53 aa, Artificial Sequence
  Synthetic Antp-AAC-11-LZ (363-399)L/G
RQIKIWFQNRRMKWKKAKLNAEKLKDFKIRGQYFARGGQVYIRQGRLALQGKT

SEQ ID NO: 6 - protein, 45 aa, Artificial Sequence
  Synthetic Antp-AAC-11-LZ (371-399
RQIKIWFQNRRMKWKKKDFKIRLQYFARGLQVYIRQLRLALQGKT

SEQ ID NO: 7 - protein, 43 aa, Artificial Sequence
  Synthetic Antp-AAC-11-LZ (371-397)
RQIKIWFQNRRMKWKKKDFKIRLQYFARGLQVYIRQLRLALQG

SEQ ID NO: 8 - protein, 37 aa, Artificial Sequence
  Synthetic Antp-AAC-11-LZ (371-391)
RQIKIWFQNRRMKWKKKDFKIRLQYFARGLQVYIRQL

SEQ ID NO: 9 - protein, 39 aa, Artificial Sequence
  Synthetic Antp-AAC-11-LZ (377-399)
RQIKIWFQNRRMKWKKLQYFARGLQVYIRQLRLALQGKT

SEQ ID NO: 10 - protein, 36 aa, Artificial Sequence
  Synthetic Antp-AAC-11-LZ (380-399)
RQIKIWFQNRRMKWKKFARGLQVYIRQLRLALQGKT

SEQ ID NO: 11 - protein, 55 aa, Artificial Sequence
  Synthetic Cter-Antp-AAC-11-LZ (363-399)
AKLNAEKLKDFKIRLQYFARGLQVYIRQLRLALQGKTRQIKIWFKKQNRRMKWKK

SEQ ID NO: 12 - protein, 23 aa, Homo sapiens
LQYFARGLQVYIRQLRLALQGKT

SEQ ID NO: 13 - protein, 33 aa, Artificial Sequence
  Synthetic Antp-AAC-11-LZ (377-399 ?385-390)
RQIKIWFQNRRMKWKKLQYFARGLLRLALQGKT

SEQ ID NO: 14 - protein, 11 aa, Artificial Sequence
  Synthetic TAT alone
YGRKKRRQRRR

SEQ ID NO: 15 - protein, 11 aa, Artificial Sequence
  Synthetic TAT (47-57) (RI)
RRRQRRKKRGY

SEQ ID NO: 16 - protein, 51 aa, Artificial Sequence
  Synthetic TAT-AAC-11-LZ (363-399)
YGRKKRRQRRRGGGAKLNAEKLKDFKIRLQYFARGLQVYIRQLRLALQGKT

SEQ ID NO: 17 - protein, 49 aa, Artificial Sequence
  Synthetic TAT-AAC-11-LZ (363-399)RI  (D-amino acids)
RRRQRRKKRGYGTKGQLALRLQRIYVQLGRAFYQLRIKFDKLKEANLKA

SEQ ID NO: 18 - protein, 53 aa, Artificial Sequence; identical to SEQ ID NO: 4
  Synthetic Fusion protein RT53

SEQ ID NO: 19 - protein, 39 aa, Artificial Sequence; identical to SEQ ID NO: 9
  Synthetic Fusion protein RT39

(19) United States
(12) Patent Application Publication
(10) Pub. No.: US 2023/0346901 A1
(43) Pub. Date: Nov. 2, 2023

(54) METHODS AND VACCINE COMPOSITIONS TO TREAT CANCERS

(71) Applicants: INSERM (INSTITUT NATIONAL DE LA SANTÉ ET DE LA RECHERCHE MÉDICALE), Paris (FR); UNIVERSITÉ PARIS CITÉ, Paris (FR)

(72) Inventors: Jean-Luc POYET, Paris (FR); Justine HABAULT, Paris Cedex 10 (FR); Marie PLA, Paris (FR)

(21) Appl. No.: 17/786,908

(22) PCT Filed: Dec. 18, 2020
(86) PCT No.: PCT/EP2020/087133

§ 371 Date: Jun. 17, 2022

(30) Foreign Application Priority Data
Dec. 19, 2019 (EP) 19306701.4

(51) Int. Cl. A61K 39/00; A61P 35/02; A61P 35/00; C12N 5/09

(52) U.S. Cl. CPC A61K 39/0011; A61P 35/02; A61P 35/00; C12N 5/0694; A61K 2039/5152; A61K 2039/55; C12N 2501/06

(57) ABSTRACT

The present invention relates to a method for obtaining a population of oncogenic cells modified comprising the following steps: i) obtaining a population of oncogenic cells from a subject suffering from a cancer; and ii) treating said cells with a fusion protein comprising an AAC-11 leucine-zipper (LZ) derived peptide which is fused to at least one heterologous polypeptide. Inventors have evaluated here the antileukemic efficacy of RT53, an anticancer peptide with potential immunological properties. Their results indicate that RT53 possesses a direct antileukemic effect, even at late stage. They also demonstrated that single injection of a vaccine consisting of leukemic blasts exposed to RT53, which induces the hallmarks of immunogenic cell death, was highly effective in preventing leukemia development in both prophylactic and therapeutic settings. The vaccine comprising RT53-treated APL cells generated long-term antileukemic protection and depletion experiments indicated that CD4+ T cells were of crucial importance for vaccine efficacy. Combined, their results provide the rational for the exploration of RT53-based therapies for the treatment of cancer, such as acute leukemia.

### FIELD OF THE INVENTION

The invention is in the field of oncology. More particularly, the invention relates to a method for obtaining a population of pluripotent cells and its use to treat cancers.

### BACKGROUND OF THE INVENTION

A major strategy in anticancer efforts is to target oncogenic signaling networks that are required for tumor cell growth and survival. However, intrinsic or acquired resistance often limits the efficacy of these targeted therapies^{1, 2}. In parallel to the development of molecular targeted agents, cancer immunotherapies are changing the therapeutic landscape for cancer patients by providing a clinically beneficial alternative to conventional treatments^{3, 4}. Despite improved molecular characterization of malignancies and development of target therapies, acute leukemia is not curable and a minority of patients survive more than 10 years after diagnosis. Among leukemia subtypes, prognosis is particularly poor in adult acute myeloid leukemia (AML)⁵. Therefore, new agents and novel regimens are clearly needed to improve outcomes for AIL patients.

AAC-11 is an antiapoptotic protein (antiapoptosis clone 11)¹⁹, also called Api5 or FIF^{20,21}. AAC-11 is a nuclear protein whose expression has been demonstrated to prevent apoptosis following growth factor deprivation^{19,22}. AAC-11 antiapoptotic action appears by the suppression of the transcription factor E2F1-induced apoptosis²⁰.

Anti-cancer vaccines can be divided into two types: therapeutic and prophylactic (preventive). Therapeutic vaccines are used to treat patients with established cancer, whereas prophylactic vaccines are used to prevent cancer from occurring. Despite considerable efforts to develop cancer vaccines, the clinical translation of cancer vaccines into efficacious therapies has been challenging with the exception of prophylactic vaccines against hepatitis B virus (HBV) and human papillomavirus (HPV), which are causes of liver and cervical cancer, respectively^{23,24}. These prophylactic vaccines have been successful because they circumvent three major challenges facing the development of therapeutic cancer vaccines: (1) low immunogenicity; (2) established disease burden; and (3) the immunosuppressive tumor microenvironment, as it is now widely accepted that tumor cells take specific steps to evade the host immune system in order to survive and metastasize²⁵. Therapeutic vaccination against established cancer is therefore much more challenging compared to prophylactic vaccination and, consequently, the only therapeutic anti-cancer vaccine that has been licensed for use in clinical practice to date is sipuleucel-T (Provenge), which is used for the treatment of prostate cancer.

The inventors have demonstrated that some peptides derived from the AAC-11 protein selectively disrupt vital cellular functions in a plurality of cancer cells, at micromolar range as described in WO2015/121496.

### SUMMARY OF THE INVENTION

The invention relates to a method for treating a cancer in a subject in need thereof comprising the following step.
i) obtaining a population of oncogenic cells from a subject suffering from a cancer;
ii) treating said oncogenic cells with a fusion protein comprising an AAC-11 leucine-zipper (LZ) derived peptide which is fused to at least one heterologous polypeptide; and
iii) administering to the subject a therapeutically effective amount of the population of the oncogenic cells modified in step ii).

In a particular embodiment, the invention is defined by claims.

### DETAILED DESCRIPTION OF THE INVENTION

By sing a well-characterized preclinical mouse model of acute promyelocytic leukemia (APL), inventors have evaluated here the antileukemic efficacy of RT53, an anticancer peptide with potential immunological properties. Their results indicate that RT53 possesses a direct antileukemic effect, even at late stage. They also demonstrated that single injection of a vaccine consisting of leukemic blasts exposed to RT53, which induces the hallmarks of immunogenic cell death, was highly effective in preventing leukemia development in both prophylactic and therapeutic settings. The vaccine comprising RT53-treated APL cells generated long-term antileukemic protection and depletion experiments indicated that CD4+ T cells were of crucial importance for vaccine efficacy. Indeed, they clearly show that the anticancer response generated by peptide-treated APL cells vaccination required the presence of both CD4+ and CD8+ T cells, indicating activation of both T cell populations. As no toxicity nor adverse side effects were detected upon vaccination, these peptides could therefore be of great interest as innovative adjuvants for human clinic. Combined, their results provide the rational for the exploration of RT53-based therapies for the treatment of cancer, such as acute leukemia.

Accordingly, in a first aspect, the invention relates to a method for obtaining a population of oncogenic cells modified comprising the following steps:
i) obtaining a population of oncogenic cells from a subject suffering from a cancer; and
ii) treating said cells with a fusion protein comprising an AAC-11 leucine-zipper (LZ) derived peptide which is fused to at least one heterologous polypeptide.

In a particular embodiment, the invention relates to an in vitro method comprising the following steps: i) obtaining a population of oncogenic cells from a subject suffering from a cancer; and ii) treating said cells with a fusion protein comprising an AAC-11 leucine-zipper (LZ) derived peptide which is fused to at least one heterologous polypeptide.

In a particular embodiment, the method according to the invention further comprises a step of inactivating the population of oncogenic cells modified with the fusion protein.

As used herein, the term “population” refers to a population of cells, wherein the majority (e.g., at least about 20%, in particular at least about 50%, more particularly at least about 70%, and even more particularly at least about 80%, and even more particularly at least about 90%) of the total number of cells have the specified characteristics of the cells of interest (e.g. pluripotency markers for iPSC, ESC or cancer stems cells defined by international stem cell initiative including at least 96 markers (Adewumi et al, Nat Biotech 2007), and gene-expression based assay (PluriTest) (F J Muller, Nature Methods 2011).

Particularly, the term “a population of oncogenic cells” refers to a population of cancer cells where the characteristics of the cells is expression of the oncogenic markers. Example of oncogenic markers expressed by oncogenic cells: increased expression of HEC1, MMP-1, hTERT, C-Myc, N-Myc, β-catenin, BMI1, Cyclin D1, HER-2, Src and Ras or BCR-ABL arrangement etc.

In a particular embodiment, the oncogenic cells are pluripotent cells.

As used herein, the term “pluripotent” refers to cells with the ability to give rise to progeny that can undergo differentiation, under appropriate conditions, into all cell types derived from the three germ layers (endoderm, mesoderm, and ectoderm) with specific cell lineages characteristics. In a particular embodiment, the pluripotent cells are selected from the group consisting of human embryonic stem cells (hESC), induced human pluripotent stem cells (hiPSC), allogeneic, xenogeneic or syngeneic/autologous stem cells.

In a particular embodiment, oncogenic cells are cancer cells drives the growth and progression of a tumour.

In a particular embodiment, the oncogenic cells are cancer stem cells also called as tumour initiating cells, or tumour stem cells.

These cancer stem cells as rare cell populations that share similar properties with normal stem cells long-term, self-renewing capacity is thought to be a determining factor in the maintenance and regrowth of the tumour. These cancer stem cells have the capacity to self-renew and to form the heterogeneous lineages of cancer cells that comprise the tumour (Xiaoyu Cheng et al 2009).

In a particular embodiment, the population of oncogenic cells is obtained from a cancer selected from the following group consisting but not limited to: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous; adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; and roblastoma, malignant; Sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malig melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

In a particular embodiment, the population of oncogenic cells is obtained from blood cancer, such as, but not limited to leukemia, lymphoma or myeloma. In a particular embodiment, the population of oncogenic cells is leukemic blast cells.

In a particular embodiment, the population of oncogenic cells is obtained from solid tumors, such as, but not limited to skin cancers, lung cancers, liver cancers, breast cancers, colon cancers, colorectal cancers, brain cancers, neuroblastoma . . . .

In a particular embodiment, the population of oncogenic cells is obtained from gene mutation-, radiation-, chemically- or virally-induced cancer, such as, but not limited to cervical, anal, and head/neck cancers from human papilloma virus (HPV)-16.

In a particular embodiment, the population of oncogenic cells is obtained from cancer stem cells.

As used herein the term “AAC-11” has its general meaning in the art and refers to the antiapoptosis clone 11 protein that is also known as Api5 or FIF. An exemplary human polypeptide sequence of AAC-11 is deposited in the GenBank database accession number: NP_001136402.1 set forth as SEQ ID NO:1.

As used herein, the “AAC-11 leucine-zipper (LZ)” refers to polypeptides and/or peptides derived from AAC-11.

In a particular embodiment, the present invention relates to a polypeptide comprising or consisting of
i) an amino acid sequence ranging from the phenylalanine residue at position 380 to the leucine residue at position 384 in SEQ ID NO:1 or,
ii) an amino acid sequence having at least 70% of identity with the amino acid sequence ranging from the phenylalanine residue at position 380 to the leucine residue at position 384 in SEQ ID NO:1, or
iii) an amino acid sequence which is a retro-inverso of the amino acid sequence ranging from the phenylalanine residue at position 380 to the leucine residue at position 384 in SEQ ID NO:1 or,
iv) an amino acid sequence which is retro-inverso of the amino acid sequence having at least 70% of identity with the amino acid sequence ranging from the phenylalanine residue at position 380 to the leucine residue at position 384 in SEQ ID NO:1,
wherein the polypeptide does not consist of the amino acid sequence SEQ ID NO:1 and does not consist of the amino acid sequence ranging from the alanine residue at position 363 to the threonine residue at position 399 in SEQ ID NO:1.

In a particular embodiment, the present invention relates to a polypeptide comprising or consisting of
i) an amino acid sequence ranging from the phenylalanine residue at position 380 to the isoleucine residue at position 388 in SEQ ID NO:1 or,
ii) an amino acid sequence having at least 70% of identity with the amino acid sequence ranging from the phenylalanine residue at position 380 to the isoleucine residue at position 388 in SEQ ID NO:1, or
iii) an amino acid sequence which is a retro-inverso of the amino acid sequence ranging from the phenylalanine residue at position 380 to the isoleucine residue at position 388 in SEQ ID NO:1 or,
iv) an amino acid sequence which is retro-inverso of the amino acid sequence having at least 70% of identity with the amino acid sequence ranging from the phenylalanine residue at position 380 to the isoleucine residue at position 388 in SEQ ID NO:1,
wherein the polypeptide does not consist of the amino acid sequence SEQ ID NO:1 and does not consist of the amino acid sequence ranging from the alanine residue at position 363 to the threonine residue at position 399 in SEQ ID NO:1.

In a particular embodiment, the present invention relates to a polypeptide comprising or consisting of
i) an amino acid sequence ranging from the phenylalanine residue at position 380 to the leucine residue at position 391 in SEQ ID NO:1 or,
ii) an amino acid sequence having at least 70% of identity with the amino acid sequence ranging from the phenylalanine residue at position 380 to the leucine residue at position 391 in SEQ ID NO:1, or
iii) an amino acid sequence which is a retro-inverso of the amino acid sequence ranging from the phenylalanine residue at position 380 to the leucine residue at position 391 in SEQ ID NO:1 or,
iv) an amino acid sequence which is retro-inverso of the amino acid sequence having at least 70% of identity with the amino acid sequence ranging from the phenylalanine residue at position 380 to the leucine residue at position 391 in SEQ ID NO:1,
wherein the polypeptide does not consist of the amino acid sequence SEQ ID NO:1 and does not consist of the amino acid sequence ranging from the alanine residue at position 363 to the threonine residue at position 399 in SEQ ID NO:1.

In a particular embodiment, the present invention relates to a polypeptide comprising or consisting of
i) an amino acid sequence ranging from the tyrosine residue at position 379 to the leucine residue at position 391 in SEQ ID NO:1 or,
ii) an amino acid sequence having at least 70% of identity with the amino acid sequence ranging from the tyrosine residue at position 379 to the leucine residue at position 391 in SEQ ID NO:1, or
iii) an amino acid sequence which is a retro-inverso of the amino acid sequence ranging from the tyrosine residue at position 379 to the leucine residue at position 391 in SEQ ID NO:1 or,
iv) an amino acid sequence which is retro-inverso having at least 70% of identity with the amino acid sequence ranging from the tyrosine residue at position 379 to the leucine residue at position 391 in SEQ ID NO:1,
wherein the polypeptide does not consist of the amino acid sequence SEQ ID NO:1 and does not consist of the amino acid sequence ranging from the alanine residue at position 363 to the threonine residue at position 399 in SEQ ID NO:1.

In a particular embodiment, the present invention relates to a polypeptide comprising or consisting of
i) an amino acid sequence ranging from the glutamine residue at position 378 to the leucine residue at position 391 in SEQ ID NO:1 or,
ii) an amino acid sequence having at least 70% of identity with the amino acid sequence ranging from glutamine residue at position 378 to the leucine residue at position 391 in SEQ ID NO:1, or
iii) an amino acid sequence which is a retro-inverso of the amino acid sequence ranging from glutamine residue at position 378 to the leucine residue at position 391 in SEQ ID NO:1 or,
iv) an amino acid sequence which is retro-inverso having at least 70% of identity with the amino acid sequence ranging from glutamine residue at position 378 to the leucine residue at position 391 in SEQ ID NO:1,
wherein the polypeptide does not consist of the amino acid sequence SEQ ID NO:1 and does not consist of the amino acid sequence ranging from the alanine residue at position 363 to the threonine residue at position 399 in SEQ ID NO:1.

In a particular embodiment, the present invention relates to a polypeptide comprising or consisting of
i) an amino acid sequence ranging from the leucine residue at position 377 to the leucine residue at position 391 in SEQ ID NO:1 or,
ii) an amino acid sequence having at least 70% of identity with the amino acid sequence ranging from the leucine residue at position 377 to the leucine residue at position 391 in SEQ ID NO:1, or
iii) an amino acid sequence which is a retro-inverso of the amino acid sequence ranging from the leucine residue at position 377 to the leucine residue at position 391 in SEQ ID NO:1 or,
iv) an amino acid sequence which is retro-inverso having at least 70% of identity with the amino acid sequence ranging from the leucine residue at position 377 to the leucine residue at position 391 in SEQ ID NO:1,
wherein the polypeptide does not consist of the amino acid sequence SEQ ID NO:1 and does not consist of the amino acid sequence ranging from the alanine residue at position 363 to the threonine residue at position 399 in SEQ ID NO:1.

In some embodiment, the present invention relates to a polypeptide comprising or consisting of
i) an amino acid sequence ranging from the lysine residue at position 371 to the glycine residue at position 397 in SEQ ID NO:1 or,
ii) an amino acid sequence having at least 70% of identity with the amino acid sequence ranging from the lysine residue at position 371 to the glycine residue at position 397 in SEQ ID NO:1, or
iii) an amino acid sequence which is a retro-inverso of the amino acid sequence ranging from the lysine residue at position 371 to the glycine residue at position 397 in SEQ ID NO:1 or,
iv) an amino acid sequence which is retro-inverso having at least 70% of identity with the amino acid sequence ranging from the lysine residue at position 371 to the glycine residue at position 397 in SEQ ID NO:1,
wherein the polypeptide does not consist of the amino acid sequence SEQ ID NO:1 and does not consist of the amino acid sequence ranging from the alanine residue at position 363 to the threonine residue at position 399 in SEQ ID NO:1.

In some embodiment, the present invention relates to a polypeptide comprising or consisting of
i) an amino acid sequence ranging from the lysine residue at position 371 to the leucine residue at position 391 in SEQ ID NO:1 or,
ii) an amino acid sequence having at least 70% of identity with the amino acid sequence ranging from the lysine residue at position 371 to the leucine residue at position 391 in SEQ ID NO:1, or
iii) an amino acid sequence which is a retro-inverso of the amino acid sequence ranging from the lysine residue at position 371 to the leucine residue at position 391 in SEQ ID NO:1 or,
iv) an amino acid sequence which is retro-inverso having at least 70% of identity with the amino acid sequence ranging from the lysine residue at position 371 to the leucine residue at position 391 in SEQ ID NO:1,
wherein the polypeptide does not consist of the amino acid sequence SEQ ID NO:1 and does not consist of the amino acid sequence ranging from the alanine residue at position 363 to the threonine residue at position 399 in SEQ ID NO:1.

In some embodiment, the present invention relates to a polypeptide comprising or consisting of
i) an amino acid sequence ranging from the phenylalanine residue at position 380 to the threonine residue at position 399 in SEQ ID NO:1 or,
ii) an amino acid sequence having at least 70% of identity with the amino acid sequence ranging from the phenylalanine residue at position 380 to the threonine residue at position 399 in SEQ ID NO:1, or
iii) an amino acid sequence which is a retro-inverso of the amino acid sequence ranging from the phenylalanine residue at position 380 to the threonine residue at position 399 in SEQ ID NO:1 or,
iv) an amino acid sequence which is retro-inverso having at least 70% of identity with the amino acid sequence ranging from the phenylalanine residue at position 380 to the threonine residue at position 399 in SEQ ID NO:1,
wherein the polypeptide does not consist of the amino acid sequence SEQ ID NO:1 and does not consist of the amino acid sequence ranging from the alanine residue at position 363 to the threonine residue at position 399 in SEQ ID NO:1.

In some embodiment, the present invention relates to a polypeptide comprising or consisting of
i) an amino acid sequence ranging from the lysine residue at position 371 to the threonine residue at position 399 in SEQ ID NO:1 or,
ii) an amino acid sequence having at least 70% of identity with the amino acid sequence ranging from the lysine residue at position 371 to the threonine residue at position 399 in SEQ ID NO:1, or
iii) an amino acid sequence which is a retro-inverso of the amino acid sequence ranging from the lysine residue at position 371 to the threonine residue at position 399 in SEQ ID NO:1 or,
iv) an amino acid sequence which is retro-inverso having at least 70% of identity with the amino acid sequence ranging from the lysine residue at position 371 to the threonine residue at position 399 in SEQ ID NO:1,
wherein the polypeptide does not consist of the amino acid sequence SEQ ID NO:1 and does not consist of the amino acid sequence ranging from the alanine residue at position 363 to the threonine residue at position 399 in SEQ ID NO:1.

In some embodiment, the present invention relates to a polypeptide comprising or consisting of
i) an amino acid sequence ranging from the leucine residue at position 377 to the threonine residue at position 399 in SEQ ID NO:1 or,
ii) an amino acid sequence having at least 70% of identity with the amino acid sequence ranging from the leucine residue at position 377 to the threonine residue at position 399 in SEQ ID NO:1, or
iii) an amino acid sequence which is a retro-inverso of the amino acid sequence ranging from the leucine residue at position 377 to the threonine residue at position 399 in SEQ ID NO:1 or,
iv) an amino acid sequence which is retro-inverso having at least 70% of identity with the amino acid sequence ranging from the leucine residue at position 377 to the threonine residue at position 399 in SEQ ID NO:1,
wherein the polypeptide does not consist of the amino acid sequence SEQ ID NO:1 and does not consist of the amino acid sequence ranging from the alanine residue at position 363 to the threonine residue at position 399 in SEQ ID NO:1.

According to the invention a first amino acid sequence having at least 70% of identity with a second amino acid sequence means that the first sequence has 70; 71; 72; 73; 74; 75; 76; 77; 78; 79; 80; 81; 82; 83; 84; 85; 86; 87; 88; 89; 90; 91; 92; 93; 94; 95; 96; 97; 98; or 99% of identity with the second amino acid sequence. Amino acid sequence identity is preferably determined using a suitable sequence alignment algorithm and default parameters, such as BLAST P (Karlin and Altschul, 1990).

The term “retro-inverso amino acid sequence” relates to an isomeric form of an amino acid sequence in which the direction of the amino acid sequence is reversed and the chirality of each amino acid residue is inverted. Retro-inverso amino acid sequence of the present invention may be composed by D-amino acids assembled in the reverse order from that of the parental amino acid sequence-sequence.

In some embodiments, the polypeptide of the invention comprises 5, 6, 7, 8, 9; 10; 11; 12; 13; 14; 15; 16; 17; 18; 19; 20; 21; 22; 23; 24; 25; 26; 27; 28; 29; 30; 31; 32; 33; 34; 35; 36; 37; 38; 39; 40; 41; 42; 43; 44; 45; 46; 47; 48; 49; 50; 51; 52; 53; 54; 55; 56; 57; 58; 59; 60; 61; 62; 63; 64; 65; 66; 67; 68; 69; 70; 71; 72; 73; 74; 75; 76; 77; 78; 79; 80; 81; 82; 83; 84; 85; 86; 87; 88; 89; 90; 91; 92; 93; 94; 95; 96; 97; 98; 99; or 100 amino acids. In some embodiments, the polypeptide of the invention comprises less than 50 amino acids. In some embodiments, the polypeptide of the invention comprises less than 30 amino acids. In some embodiments, the polypeptide of the invention comprises less than 25 amino acids. In some embodiments, the polypeptide of the invention comprises less than 20 amino acids. In some embodiments, the polypeptide of the invention comprises less than 15 amino acids.

As used herein, the term “fusion protein” refers to the polypeptide according to the invention that is fused directly or via a spacer to at least one heterologous polypeptide.

Typically, the polypeptides or fusion proteins of the invention can be produced by any technique known per se in the art, such as, without limitation, any chemical, biological, genetic or enzymatic technique, either alone or in combination. Such methods are described in WO2015/121496.

According to the invention, the fusion protein comprises the polypeptide as described above that is fused either directly or via a spacer at its C-terminal end to the N-terminal end of the heterologous polypeptide, or at its N-terminal end to the C-terminal end of the heterologous polypeptide.

As used herein, the term “directly” means that the (first or last) amino acid at the terminal end (N or C-terminal end) of the polypeptide is fused to the (first or last) amino acid at the terminal end (N or C-terminal end) of the heterologous polypeptide.

In other words, in this embodiment, the last amino acid of the C-terminal end of said polypeptide is directly linked by a covalent bond to the first amino acid of the N-terminal end of said heterologous polypeptide, or the first amino acid of the N-terminal end of said polypeptide is directly linked by a covalent bond to the last amino acid of the C-terminal end of said heterologous polypeptide.

As used herein, the term “spacer” refers to a sequence of at least one amino acid that links the polypeptide of the invention to the heterologous polypeptide. Such a spacer may be useful to prevent steric hindrances.

In some embodiments, the heterologous polypeptide is a cell-penetrating peptide, a Transactivator of Transcription (TAT) cell penetrating sequence, a cell permeable peptide or a membranous penetrating sequence.

As used herein, the term “cell-penetrating peptides” are well known in the art and refers to cell permeable sequence or membranous penetrating sequence such as penetratin, TAT mitochondrial penetrating sequence and compounds (Bechara and Sagan, 2013; Jones and Sayers, 2012; Khafagy el and Morishita, 2012; Malhi and Murthy, 2012).

In a particular embodiment, the heterologous polypeptide is an internalization sequence derived either from the homeodomain of Drosophila Antennapedia/Penetratin (Antp) protein (amino acids 43-58; SEQ ID NO: 2) or the Transactivator of Transcription (TAT) cell penetrating sequences (SEQ TD NO:14).

In a particular embodiment, one, two or three glycine residue are added at the C-terminal end of the TAT cell penetrating sequences (SEQ TD NO: 14).

In some embodiments, the fusion protein of the present invention comprises or consists of a sequence selected from the group consisting of SEQ ID NO:4, SEQ ID NO:6; SEQ ID NO:7, SEQ ID NO:8, SEQ ID NO:9, SEQ ID NO:10; SEQ ID NO:11; SEQ ID NO:13, SEQ ID NO:14; SEQ ID NO:16; SEQ ID NO:17; SEQ ID NO:18 and SEQ ID NO:19.

In a particular embodiment, the fusion protein comprises or consists of a sequence SEQ ID NO: 18.

In a particular embodiment, the fusion protein comprises or consists of a sequence SEQ ID NO: 19.

TABLE 1
amino acid sequence of AAC-11 LZ-derived
peptides and fusion proteins

| Name | Sequences |
|---|---|
| AAC-11 | SEQ ID NO: 1 |
| isoform a | MPTVEELYRNYGILADATEQVGQHKDAYQVILDG |
| NP_001136402.1 | VKGGTKEKRLAAQFIPKFFKHFPELADSAINAQL |
| | DLCEDEDVSIRRQAIKELPQFATGENLPRVADIL |
| | TQLLQTDDSAEFNLVNNALLSIFKMDAKGTLGGL |
| | FSQILQGEDIVRERAIKFLSTKLKTLPDEVLTKE |
| | VEELILTESKKVLEDVTGEEFVLFMKILSGLKSL |
| | QTVSGRQQLVELVAEQADLEQTFNPSDPDCVDRL |
| | LQCTRQAVPLFSKNVHSTRFVTYFCEQVLPNLGT |
| | LTTPVEGLDIQLEVLKLLAEMSSFCGDMEKLETN |
| | LRKLFDKLLEYMPLPPEEAENGENAGNEEPKLQF |
| | SYVECLLYSFHQLGRKLPDFLTAKLNAEKLKDFK |
| | IRLQYFARGLQVYIRQLRLALQGKTGEALKTEEN |
| | KIKVVALKITNNINVLIKDLFHIPPSYKSTVTLS |
| | WKPVQKVEIGQKRASEDTTSGSPPKKSSAGPKRD |
| | ARQIYNPPSGKYSSNLGNFNYEQRGAFRGSRGGR |
| | GWGTRGNRSRGRLY |
| Penetratin | SEQ ID NO: 2 |
| alone | RQIKIWFQNRRMKWKK |
| AAC-11-LZ | SEQ ID NO: 3 |
| (363-399) | AKLNAEKLKDFKIRLQYFARGLQVYIRQLRLALQ |
| | GKT |
| Antp-AAC-11- | SEQ ID NO: 4 |
| LZ (363-399) | RQIKIWFQNRRMKWKKAKLNAEKLKDFKIRLQYF |
| | ARGLQVYIRQLRLALQGKT |
| Antp-AAC-11- | SEQ ID NO: 5 |
| LZ (363- | RQIKIWFQNRRMKWKKAKLNAEKLKDFKIRGQYF |
| 399)L/G | ARGGQVYIRQGRLALQGKT |
| Antp-AAC-11- | SEQ ID NO: 6 |
| LZ (371-399) | RQIKIWFQNRRMKWKKKDFKIRLQYFARGLQVYI |
| | RQLRLALQGKT |
| Antp-AAC-11- | SEQ ID NO: 7 |
| LZ (371-397) | RQIKIWFQNRRMKWKKKDFKIRLQYFARGLQVYI |
| | RQLRLALQG |
| Antp-AAC-11- | SEQ ID NO: 8 |
| LZ (371-391) | RQIKIWFQNRRMKWKKKDFKIRLQYFARGLQVYI |
| | RQL |
| Antp-AAC-11- | SEQ ID NO: 9 |
| LZ (377-399) | RQIKIWFQNRRMKWKKLQYFARGLQVYIRQLRLA |
| | LQGKT |
| Antp-AAC-11- | SEQ ID NO: 10 |
| LZ (380-399) | RQIKIWFQNRRMKWKKFARGLQVYIRQLRLALQG |
| | KT |
| Cter-Antp- | SEQ ID NO: 11 |
| AAC-11-LZ | AKLNAEKLKDFKIRLQYFARGLQVYIRQLRLALQ |
| (363-399) | GKTRQIKIWFKKQNRRMKWKK |
| AAC-11-LZ | SEQ ID NO: 12 |
| (377-399) | LQYFARGLQVYIRQLRLALQGKT |
| Antp-AAC-11- | SEQ ID NO: 13 |
| LZ (377-399 | RQIKIWFQNRRMKWKKLQYFARGLLRLALQGKT |
| Δ385-390) | |
| TAT alone | SEQ ID NO: 14 |
| | YGRKKRRQRRR |
| TAT (47-57) | SEQ ID NO: 15 |
| (RI) | RRRQRRKKRGY (D-amino acids) |
| TAT-AAC-11- | SEQ ID NO: 16 |
| LZ (363-399) | YGRKKRRQRRRGGGAKLNAEKLKDFKIRLQYFA |
| | RGLQVYIRQLRLALQGKT |
| TAT-AAC-11- | SEQ ID NO: 17 |
| LZ (363-399)RI | RRRQRRKKRGYGTKGQLALRLQRIYVQLGRAFY |
| (D-amino | QLRIKFDKLKEANLKA |
| acids) | |
| Fusion pro- | SEQ ID NO: 18 |
| tein RT53 | RQIKIWFQNRRMKWKKAKLNAEKLKDFKIRLQY |
| | FARGLQVYIRQLRLALQGKT |
| Fusion pro- | SEQ ID NO: 19 |
| tein RT39 | RQIKIWFQNRRMKWKKLQYFARGLQVYIRQLRL |
| | ALQGKT |

The population of oncogenic cells obtained according to the method of the present invention is inactivated with a mutagenic agent.

Accordingly, the invention relates to a method for obtaining a population of oncogenic cells modified comprising the following steps:
i) obtaining a population of oncogenic cells from a subject suffering from a cancer; and
ii) treating said cells with a fusion protein comprising an AAC-11 leucine-zipper (LZ) derived peptide which is fused to at least one heterologous polypeptide and/or
iii) inactivating said population obtained at step ii) with a mutagenic agent.

In a particular embodiment, the mutagenic agent is selected from the group consisting of chemical mutagenic agents and radiation mutagenic agent (X-Ray, UV radiation).

In particular, the mutagenic agent is selected from the group consisting of ENU, reactive oxygen species, deaminating agents, polycyclic aromatic hydrocarbons, aromatic amines and sodium azide.

The population of oncogenic cells that used in the present invention is inactivated. As used herein, the term “inactivated”, and grammatical variants thereof, refers to a cell (e.g., a pluripotent cell) that is alive but has been rendered incapable of proliferation (i.e., mitotically inactivated). The skilled in the art may use techniques that are known in the art including, but not limited to exposure to chemical agents, irradiation and/or lyophilization. Pluripotent cells can be inactivated such that upon administration to a subject the pluripotent cells are incapable of dividing and thus cannot form a pluripotent tissue in the subject. It is understood that in the context of a plurality of cells, not every cell needs to be incapable of proliferation. Thus, as used herein the phrase “inactivated to an extent sufficient to prevent tissue formation in the subject” refers to a degree of inactivation in the population as a whole such that after administration to a subject, a pluripotent tissue does not form since the irradiated pluripotent cells did not divide anymore as confirmed by in vitro culture. It is to be noted that, even if a one or more cells in the plurality of cells are in fact capable of proliferation in the subject, it is postulated that the immune system of the host will destroy those cells before a pluripotent tissue could form. Such inability of proliferation and tissue formation may be confirmed by testing in mice having a functional and a non-functional immune system.

In some embodiments, the “inactivated” cell is a killed cell. In another embodiment, the inactivated cell is a whole cellular lysate, pluripotent cells or oganoid derived exosomes, enriched cancer neo-antigens, a whole purified cancer neo-antigens, DNA, mRNA and protein extracts, a whole cells suspension that has been lyophilized, a fraction of a cellular lysate such as a membrane fraction, a cytoplasmic fraction, or a combination thereof. Inactivated pluripotent cells remain capable of stimulating immune response when the vaccination of mice is carried out with pluripotent cells unmodified (non-treated with a peptide derived from AAC11). This vaccination is able to induce efficient immune and anti-tumoral responses against carcinoma without evidence of side effects and autoimmune diseases.

Typically, to inactivate the oncogenic cells, they can be exposed to lethal doses of radiation, (e.g., 5 to 100 Gy single fraction). The precise radiation dose delivered to the pluripotent cells and length of dose are not critical so long as the cells are rendered nonviable.

The recovery step of the method includes one (or multiple) step(s) of washing the cell culture and resuspending the cells in any appropriate medium such as any clinical grade cell media. The conditioning of the cells may include freezing or lyophilizing the cells, in order to be able to store the cell composition before use.

In a particular embodiment, the method according to the invention is suitable to obtain a vaccine composition.

In a particular embodiment, the population of oncogenic cells obtained according to the method of the present invention for use in a vaccine composition.

In a particular embodiment, the invention refers to a population of oncogenic cells obtained according to the method of the present invention for use in a therapeutic vaccine composition.

In a particular embodiment, the population of oncogenic cells obtained according to the method of the present invention for use in the treatment of a cancer in a subject in need thereof.

In a particular embodiment, the population of oncogenic cells obtained according to the method of the present invention for use in the treatment of cancer resistant in a subject in need thereof.

In a second aspect, the invention relates to a vaccine composition comprising a population of oncogenic cells modified according to the invention.

In a particular embodiment, the invention relates to a vaccine composition comprising a population of oncogenic cells modified according to the invention and an adjuvant.

In a particular embodiment, the invention relates to a vaccine composition comprising a population of oncogenic cells treated with a fusion protein comprising an AAC-11 leucine-zipper (LZ) derived peptide which is fused to at least one heterologous polypeptide.

In a particular embodiment, the vaccine composition is a therapeutic vaccine composition.

As used herein, the term “therapeutic vaccine composition” refers to a vaccine composition enabling to treat subjects with established cancer.

In a particular embodiment, the vaccine composition according to the invention for use in the treatment of a cancer in a subject in need thereof.

In a particular embodiment, the vaccine composition according to the invention for use in the treatment of a resistant cancer in a subject in need thereof.

In a particular embodiment, the invention relates to a method for treating a cancer in a subject in need thereof comprising a step of administering the subject a therapeutically effective amount of a population of oncogenic cells modified according to the present invention.

Thus, the invention refers to a method for treating a cancer in a subject in need thereof comprising the following steps:
i) obtaining a population of oncogenic cells from a subject suffering from a cancer; and
ii) treating said oncogenic cells with a fusion protein comprising an AAC-11 leucine-zipper (LZ) derived peptide which is fused to at least one heterologous polypeptide; and
iii) administering to the subject a therapeutically effective amount of the population of oncogenic cells modified in step ii).

In a particular embodiment, the invention relates to a method for treating a cancer in a subject in need thereof comprising administering the subject with a therapeutically effective amount of the vaccine composition according to the invention.

In a particular embodiment, the invention relates to a method for treating a resistant cancer in a subject in need thereof comprising a step of administering the subject a therapeutically effective amount of a population of oncogenic cells modified according to the present invention.

In a particular embodiment, the invention relates to a method for treating a resistant cancer in a subject in need thereof comprising administering the subject with a therapeutically effective amount of the vaccine composition according to the invention.

As used herein, the terms “treating” or “treatment” refer to curative or disease modifying treatment, including treatment of subject who are ill or have been diagnosed as suffering from a disease or medical condition (i.e cancer), and includes suppression of clinical relapse. The treatment may be administered to a subject having a medical disorder (i.e cancer), in order to cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a subject during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a subject during treatment of an illness, e.g., to keep the subject in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., pain, disease manifestation, etc.]).

As used herein, the term “cancer” refers to a malignant growth or tumour resulting from an uncontrolled division of cells. The term “cancer” includes primary tumors and metastatic tumors as defined above.

As used herein, the term “resistant cancer” refers to a cancer that does not respond to treatment notably to convention therapies (chemotherapy, radiotherapy etc). In a particular embodiment, the cancer is resistant at the beginning of treatment. In another embodiment, the cancer become resistant during treatment, such cancer is called refractory cancer.

In a particular embodiment, the cancer is a hematological cancer. As used herein, the term “hematological cancer” also known as “blood cancer” refers to cancer that begins in blood-forming tissue, such as the bone marrow, or in the cells of the immune system. There are three main types of blood cancers: leukemia, lymphoma and myeloma. Hematological malignancies derive from either of the two major blood cell lineages: myeloid and lymphoid cell lines. The myeloid cell line normally produces granulocytes, erythrocytes, thrombocytes, macrophages and mast cells; the lymphoid cell line produces B, T, NK and plasma cells. Lymphomas, lymphocytic leukemias, and myeloma are from the lymphoid line, while acute and chronic myelogenous leukemia, myelodysplastic syndromes and myeloproliferative diseases are myeloid in origin.

In a particular embodiment, the hematological cancer is selected from the group consisting of but not limited to: Acute lymphoblastic leukemia (ALL), Acute myelogenous leukemia (AML), Chronic lymphocytic leukemia (CLL), small lymphocytic lymphoma (SLL), Chronic myelogenous leukemia (CML), Acute monocytic leukemia (AMoL), acute promyelocytic leukaemia (APL), hairy cell leukaemia (HCL), large granular lymphocytic leukaemia (LGL), t-cell acute lymphoblastic leukaemia (T-ALL), chronic myelomonocytic leukaemia (CMML), Lymphomas, Hodgkin's lymphomas (all four subtypes), Non-Hodgkin's lymphomas (all subtypes), multiple myeloma.

In a particular embodiment, the population of oncogenic cells modified according to the present invention and/or the vaccine composition comprising said population of oncogenic cells modified for use in the treatment of a hematological cancer such as leukemia.

In a particular embodiment, the population of oncogenic cells modified according to the present invention and/or the vaccine composition comprising said population of oncogenic cells modified for use in the treatment of acute myelogenous leukemia (AML).

In a particular embodiment, the population of oncogenic cells modified according to the present invention and/or the vaccine composition comprising said population of oncogenic cells modified for use in the treatment of resistant acute myelogenous leukemia (AML).

In a particular embodiment, the population of oncogenic cells modified according to the present invention and/or the vaccine composition comprising said population of oncogenic cells modified for use in the treatment of melanoma.

As used herein, the term “subject” denotes a mammal, such as a rodent, a feline, a canine, and a primate. Particularly, the subject according to the invention is a human. More particularly, the subject according to the invention has a cancer as described above. More particularly, the subject according to the invention has a cancer resistant. In a particular embodiment, the subject according to the invention has acute leukemia. In a particular embodiment, the subject according to the invention has melanoma.

As used herein, the term “adjuvant” means a compound that is administered for the purpose of enhancing immunogenicity of an antigen or a vaccine, and in the present specification, it is expressed as “immunostimulatory adjuvants” or merely “adjuvant”. Herein “immunostimulatory adjuvant” are administered to an subject to stimulate an immune response against an antigen which can be derived from bacterial cells, mycobacterial cells, or virus wherein said bacterial cells can be killed, live and/or attenuated, for example, BCG, which is a live attenuated bacterial vaccine.

Non-limiting examples of some commonly used vaccine adjuvants include insoluble aluminum compounds, calcium phosphate, liposomes, Virosomes™, ISCOMS®, microparticles (e.g., PLG), emulsions (e.g., MF59, Montanides), virus-like particles & viral vectors. PolyICLC (a synthetic complex of carboxymethylcellulose, polyinosinic-polycytidylic acid, and poly-L-lysine double-stranded RNA), which is a TLR3 agonist. It will be understood that other TLR agonists may also be used (e.g. TLR4 agonists, TLR5 agonists, TLR7 agonists, TLR9 agonists), or any combinations or modifications thereof.

Examples of adjuvants that may be effective include but are not limited to: aluminum hydroxide, N-acetyl-muramyl-L-threonyl-D-isoglutamine (thr-MDP), N-acetyl-nor-muramyl-L-alanyl-D-isoglutamine, MTP-PE, MF59 and RIBI (also known as SAS), which contains Monophosphoryl Lipid A (MPL)(detoxified endotoxin) from Salmonella minnesota and synthetic Trehalose Dicorynomycolate (TDM) in 2% oil (squalene)-Tween® 80-water (see for review Pulendran and Ahmed, 2011). Other examples of adjuvants include DDA (dimethyldioctadecylammonium bromide), Freund's complete and incomplete adjuvants and QuilA. In addition, immune modulating substances such as lymphokines (e.g., IFN-[gamma], IL-2 and IL-12) or synthetic IFN-[gamma] inducers such as poly I:C can be used in combination with adjuvants described herein.

In a particular embodiment, the invention relates to a vaccine composition comprising a population of oncogenic cells modified according to the invention and an adjuvant, wherein the adjuvant is a fusion protein comprising an AAC-11 leucine-zipper (LZ) derived peptide which is fused to at least one heterologous polypeptide.

In a particular embodiment, the adjuvant is RT53 or RT39.

As used herein the terms “administering” or “administration” refer to the act of injecting or otherwise physically delivering a substance as it exists outside the body (e.g., vaccine composition) into the subject, such as by mucosal, intradermal, intravenous, subcutaneous, intramuscular delivery and/or any other method of physical delivery described herein or known in the art. When a disease, or a symptom thereof, is being treated, administration of the substance typically occurs after the onset of the disease or symptoms thereof. When a disease or symptoms thereof, are being prevented, administration of the substance typically occurs before the onset of the disease or symptoms thereof.

As used herein, the term “administration simultaneously” refers to administration of 2 active ingredients by the same route and at the same time or at substantially the same time. The term “administration separately” refers to an administration of 2 active ingredients at the same time or at substantially the same time by different routes. The term “administration sequentially” refers to an administration of 2 active ingredients at different times, the administration route being identical or different.

By a “therapeutically effective amount” is meant a sufficient amount of a vaccine composition for use in a method for treating a cancer in a subject in need thereof at a reasonable benefit/risk ratio applicable to any medical treatment. It will be understood that the total daily usage of the compounds and compositions of the present invention will be decided by the attending physician within the scope of sound medical judgment. The specific therapeutically effective dose level for any particular subject will depend upon a variety of factors including the age, body weight, general health, sex and diet of the subject; the time of administration, route of administration, and rate of excretion of the specific compound employed; the duration of the treatment; drugs used in combination or coincidental with the specific polypeptide employed; and like factors well known in the medical arts. For example, it is well known within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Typically, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the subject to be treated. A medicament typically contains from about 0.01 mg to about 500 mg of the active ingredient, typically from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

In a third aspect, the invention relates to a combined preparation of i) a population of oncogenic cells and ii) a fusion protein comprising an AAC-11 leucine-zipper (LZ) derived peptide which is fused to at least one heterologous polypeptide for use by simultaneous, separate or sequential administration in the treatment a cancer in a subject.

In a particular embodiment, the invention relates to i) a population of oncogenic cells modified according to the invention and ii) a classical treatment used as a combined preparation for use in the treatment of a cancer.

In a particular embodiment, the invention relates to i) a vaccine composition comprising a population of oncogenic cells treated with a fusion protein comprising an AAC-11 leucine-zipper (LZ) derived peptide which is fused to at least one heterologous polypeptide and ii) a classical treatment used as a combined preparation for use in the treatment of a cancer.

In a particular embodiment, the invention relates to a combined preparation of i) a population of oncogenic cells and ii) a fusion protein comprising an AAC-11 leucine-zipper (LZ) derived peptide which is fused to at least one heterologous polypeptide for use by simultaneous, separate or sequential administration in the treatment of resistant cancer in a subject.

In a particular embodiment, the invention relates to i) a population of oncogenic cells modified according to the invention and ii) a classical treatment used as a combined preparation for use in the treatment of a resistant cancer.

In a particular embodiment, the invention relates to i) a vaccine composition comprising a population of oncogenic cells treated with a fusion protein comprising an AAC-11 leucine-zipper (LZ) derived peptide which is fused to at least one heterologous polypeptide and ii) a classical treatment used as a combined preparation for use in the treatment of resistant cancer.

In a particular embodiment, the invention relates to i) a vaccine composition comprising a population of oncogenic cells treated with a fusion protein comprising an AAC-11 leucine-zipper (LZ) derived peptide which is fused to at least one heterologous polypeptide and ii) an adjuvant as described above used as a combined preparation for use in the treatment of cancer.

In a particular embodiment, the invention relates to i) a vaccine composition comprising a population of oncogenic cells treated with a fusion protein comprising an AAC-11 leucine-zipper (LZ) derived peptide which is fused to at least one heterologous polypeptide and ii) an adjuvant as described above used as a combined preparation for use in the treatment of resistant cancer.

As used herein, the terms “combined treatment”, “combined therapy” or “therapy combination” refer to a treatment that uses more than one medication.

As used herein, the term “administration simultaneously” refers to administration of 2 active ingredients by the same route and at the same time or at substantially the same time. The term “administration separately” refers to an administration of 2 active ingredients at the same time or at substantially the same time by different routes. The term “administration sequentially” refers to an administration of 2 active ingredients at different times, the administration route being identical or different.

In a particular embodiment, the population of oncogenic cells modified according to the invention is combined with a classical treatment for use in the treatment of a cancer or a resistant cancer.

In a particular embodiment, the vaccine composition according to the invention is combined with a classical treatment for use in the treatment a cancer or a resistant cancer.

As used herein, the term “classical treatment” refers to treatments well known in the art and used to treat a cancer. In the context of the invention, the classical treatment refers to radiation therapy, immunotherapy or chemotherapy.

In a particular embodiment, the invention relates to i) a population of oncogenic cells modified according to the invention and ii) a radiation therapy used as a combined preparation for use in the treatment of a cancer and/or resistant cancer.

In a particular embodiment, the invention relates to i) a vaccine composition comprising a population of oncogenic cells treated with a fusion protein comprising an AAC-11 leucine-zipper (LZ) derived peptide which is fused to at least one heterologous polypeptide and ii) a radiation therapy used as a combined preparation for use in the treatment of a cancer and/or resistant cancer

As used herein, the term “radiation therapy” or “radiotherapy” have their general meaning in the art and refers the treatment of cancer with ionizing radiation. Ionizing radiation deposits energy that injures or destroys cells in the area being treated (the target tissue) by damaging their genetic material, making it impossible for these cells to continue to grow. One type of radiation therapy commonly used involves photons, e.g. X-rays. Depending on the amount of energy they possess, the rays can be used to destroy cancer cells on the surface of or deeper in the body. The higher the energy of the x-ray beam, the deeper the x-rays can go into the target tissue. Linear accelerators and betatrons produce x-rays of increasingly greater energy. The use of machines to focus radiation (such as x-rays) on a cancer site is called external beam radiation therapy. Gamma rays are another form of photons used in radiation therapy. Gamma rays are produced spontaneously as certain elements (such as radium, uranium, and cobalt 60) release radiation as they decompose, or decay. In some embodiments, the radiation therapy is external radiation therapy. Examples of external radiation therapy include, but are not limited to, conventional external beam radiation therapy; three-dimensional conformal radiation therapy (3D-CRT), which delivers shaped beams to closely fit the shape of a tumor from different directions; intensity modulated radiation therapy (IMRT), e.g., helical tomotherapy, which shapes the radiation beams to closely fit the shape of a tumor and also alters the radiation dose according to the shape of the tumor; conformal proton beam radiation therapy; image-guided radiation therapy (IGRT), which combines scanning and radiation technologies to provide real time images of a tumor to guide the radiation treatment; intraoperative radiation therapy (IORT), which delivers radiation directly to a tumor during surgery; stereotactic radiosurgery, which delivers a large, precise radiation dose to a small tumor area in a single session; hyperfractionated radiation therapy, e.g., continuous hyperfractionated accelerated radiation therapy (CHART), in which more than one treatment (fraction) of radiation therapy are given to a subject per day; and hypofractionated radiation therapy, in which larger doses of radiation therapy per fraction is given but fewer fractions.

In a particular embodiment, the invention relates to i) a population of oncogenic cells modified according to the invention and ii) a chemotherapy used as a combined preparation for use in the treatment of a cancer and/or resistant cancer.

In a particular embodiment, the invention relates to i) a vaccine composition comprising a population of oncogenic cells treated with a fusion protein comprising an AAC-11 leucine-zipper (LZ) derived peptide which is fused to at least one heterologous polypeptide and ii) a chemotherapy used as a combined preparation for use in the treatment of a cancer and/or resistant cancer.

As used herein, the term “chemotherapy” refers to use of chemotherapeutic agents to treat a subject. As used herein, the term “chemotherapeutic agent” refers to chemical compounds that are effective in inhibiting tumor growth.

Examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaorarnide and trimethylolomelamine; acetogenins (especially bullatacin and bullatacinone); a carnptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CBI-TMI); eleutherobin; pancratistatin; a sarcodictyin; spongistatin; nitrogen mustards such as chlorambucil, chlornaphazine, cholophosphamide, estrarnustine, ifosfamide, mechlorethamine, mechlorethamine oxide hydrochloride, melphalan, novembichin, phenesterine, prednimus tine, trofosfamide, uracil mustard; nitrosureas such as carmustine, chlorozotocin, fotemustine, lomustine, nimustine, ranimustine; antibiotics such as the enediyne antibiotics (e.g. calicheamicin, especially calicheamicin (11 and calicheamicin 211, see, e.g., Agnew Chem Intl. Ed. Engl. 33: 183-186 (1994); dynemicin, including dynemicin A; an esperamicin; as well as neocarzinostatin chromophore and related chromoprotein enediyne antiobiotic chromomophores), aclacinomysins, actinomycin, authramycin, azaserine, bleomycins, cactinomycin, carabicin, canninomycin, carzinophilin, chromomycins, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idanrbicin, marcellomycin, mitomycins, mycophenolic acid, nogalarnycin, olivomycins, peplomycin, potfiromycin, puromycin, quelamycin, rodorubicin, streptomgrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophospharnide glycoside; aminolevulinic acid; amsacrine; bestrabucil; bisantrene; edatraxate; defo famine; demecolcine; diaziquone; elfornithine; elliptinium acetate; an epothilone; etoglucid; gallium nitrate; hydroxyurea; lentinan; lonidamine; maytansinoids such as maytansine and ansamitocins; mitoguazone; mitoxantrone; mopidamol; nitracrine; pento statin; phenamet; pirarubicin; podophyllinic acid; 2-ethylhydrazide; procarbazine; PSK®; razoxane; rhizoxin; sizofiran; spirogennanium; tenuazonic acid; triaziquone; 2,2′,2″-trichlorotriethylarnine; trichothecenes (especially T-2 toxin, verracurin A, roridinA and anguidine); urethan; vindesine; dacarbazine; mannomustine; mitobromtol; mitolactol; pipobroman; gacytosine; arabinoside (“Ara-C”); cyclophosphamide; thiotepa; taxoids, e.g. paclitaxel (TAXOL®, Bristol-Myers Squibb Oncology, Princeton, N.J.) and doxetaxel (TAXOTERE®, Rhone-Poulenc Rorer, Antony, France); chlorambucil; gemcitabine; 6-thioguanine; mercaptopurine; methotrexate; platinum analogs such as cisp latin and carbop latin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-1 1; topoisomerase inhibitor RFS 2000; difluoromethylornithine (DMFO); retinoic acid; capecitabine; and pharmaceutically acceptable salts, acids or derivatives of any of the above. Also included in this definition are antihormonal agents that act to regulate or inhibit honnone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and pharmaceutically acceptable salts, acids or derivatives of any of the above.

In a particular embodiment, the cancer (such as acute leukemia) is resistant to a treatment with an immune checkpoint inhibitor.

In a particular embodiment, the invention relates to i) a population of oncogenic cells modified according to the invention and ii) an immune checkpoint inhibitor used as a combined preparation for use in the treatment of a cancer and/or resistant cancer.

In a particular embodiment, the invention relates to i) a vaccine composition comprising a population of oncogenic cells treated with a fusion protein comprising an AAC-11 leucine-zipper (LZ) derived peptide which is fused to at least one heterologous polypeptide and ii) an immune checkpoint inhibitor used as a combined preparation for use in the treatment of a cancer and/or resistant cancer.

As used herein, the term “immune checkpoint inhibitor” refers to molecules that totally or partially reduce, inhibit, interfere with or modulate one or more immune checkpoint proteins. As used herein, the term “immune checkpoint protein” has its general meaning in the art and refers to a molecule that is expressed by T cells in that either turn up a signal (stimulatory checkpoint molecules) or turn down a signal (inhibitory checkpoint molecules). Immune checkpoint molecules are recognized in the art to constitute immune checkpoint pathways similar to the CTLA-4 and PD-1 dependent pathways (see e.g. Pardoll, 2012. Nature Rev Cancer 12:252-264; Mellman et al., 2011. Nature 480:480-489). Examples of stimulatory checkpoint include CD27 CD28 CD40, CD122, CD137, OX40, GITR, and ICOS. Examples of inhibitory checkpoint molecules include A2AR, B7-H3, B7-H4, BTLA, CTLA-4, CD277, IDO, KIR, PD-1, LAG-3, TIM-3 and VISTA. The Adenosine A2A receptor (A2AR) is regarded as an important checkpoint in cancer therapy because adenosine in the immune microenvironment, leading to the activation of the A2a receptor, is negative immune feedback loop and the tumor microenvironment has relatively high concentrations of adenosine. B7-H3, also called CD276, was originally understood to be a co-stimulatory molecule but is now regarded as co-inhibitory. B7-H4, also called VTCN1, is expressed by tumor cells and tumor-associated macrophages and plays a role in tumour escape. B and T Lymphocyte Attenuator (BTLA) and also called CD272, has HVEM (Herpesvirus Entry Mediator) as its ligand. Surface expression of BTLA is gradually downregulated during differentiation of human CD8+ T cells from the naive to effector cell phenotype, however tumor-specific human CD8+ T cells express high levels of BTLA. CTLA-4, Cytotoxic T-Lymphocyte-Associated protein 4 and also called CD152. Expression of CTLA-4 on Treg cells serves to control T cell proliferation. IDO, Indoleamine 2,3-dioxygenase, is a tryptophan catabolic enzyme. A related immune-inhibitory enzymes. Another important molecule is TDO, tryptophan 2,3-dioxygenase. IDO is known to suppress T and NK cells, generate and activate Tregs and myeloid-derived suppressor cells, and promote tumour angiogenesis. KIR, Killer-cell Immunoglobulin-like Receptor, is a receptor for MHC Class I molecules on Natural Killer cells. LAG3, Lymphocyte Activation Gene-3, works to suppress an immune response by action to Tregs as well as direct effects on CD8+ T cells. PD-1, Programmed Death 1 (PD-1) receptor, has two ligands, PD-L1 and PD-L2. This checkpoint is the target of Merck & Co.'s melanoma drug Keytruda, which gained FDA approval in September 2014. An advantage of targeting PD-1 is that it can restore immune function in the tumor microenvironment. TIM-3, short for T-cell Immunoglobulin domain and Mucin domain 3, expresses on activated human CD4+ T cells and regulates Th1 and Thl7 cytokines. TIM-3 acts as a negative regulator of Th1/Tc1 function by triggering cell death upon interaction with its ligand, galectin-9. VISTA, Short for V-domain Ig suppressor of T cell activation, VISTA is primarily expressed on hematopoietic cells so that consistent expression of VISTA on leukocytes within tumors may allow VISTA blockade to be effective across a broad range of solid tumors. Tumor cells often take advantage of these checkpoints to escape detection by the immune system. Thus, inhibiting a checkpoint protein on the immune system may enhance the anti-tumor T-cell response.

In some embodiments, an immune checkpoint inhibitor refers to any compound inhibiting the function of an immune checkpoint protein. Inhibition includes reduction of function and full blockade. In some embodiments, the immune checkpoint inhibitor could be an antibody, synthetic or native sequence peptides, small molecules or aptamers which bind to the immune checkpoint proteins and their ligands.

In a particular embodiment, the immune checkpoint inhibitor is an antibody.

Typically, antibodies are directed against A2AR, B7-H3, B7-H4, BTLA, CTLA-4, CD277, IDO, KIR, PD-1, LAG-3, TIM-3 or VISTA.

In a particular embodiment, the immune checkpoint inhibitor is an anti-PD-1 antibody such as described in WO2011082400, WO2006121168, WO2015035606, WO2004056875, WO2010036959, WO2009114335, WO2010089411, WO2008156712, WO2011110621, WO2014055648 and WO2014194302. Examples of anti-PD-1 antibodies which are commercialized: Nivolumab (Opdivo®, BMS), Pembrolizumab (also called Lambrolizumab, KEYTRUDA® or MK-3475, MERCK).

In some embodiments, the immune checkpoint inhibitor is an anti-PD-L1 antibody such as described in WO2013079174, WO2010077634, WO2004004771, WO2014195852, WO2010036959, WO2011066389, WO2007005874, WO2015048520, U.S. Pat. No. 8,617,546 and WO2014055897. Examples of anti-PD-L1 antibodies which are on clinical trial: Atezolizumab (MPDL3280A, Genentech/Roche), Durvalumab (AZD9291, AstraZeneca), Avelumab (also known as MSB0010718C, Merck) and BMS-936559 (BMS).

In some embodiments, the immune checkpoint inhibitor is an anti-PD-L2 antibody such as described in U.S. Pat. Nos. 7,709,214, 7,432,059 and 8,552,154.

In the context of the invention, the immune checkpoint inhibitor inhibits Tim-3 or its ligand.

In a particular embodiment, the immune checkpoint inhibitor is an anti-Tim-3 antibody such as described in WO03063792, WO2011155607, WO2015117002, WO2010117057 and WO2013006490.

In some embodiments, the immune checkpoint inhibitor is a small organic molecule.

The term “small organic molecule” as used herein, refers to a molecule of a size comparable to those organic molecules generally used in pharmaceuticals. The term excludes biological macro molecules (e. g. proteins, nucleic acids, etc.). Typically, small organic molecules range in size up to about 5000 Da, more preferably up to 2000 Da, and most preferably up to about 1000 Da.

Typically, the small organic molecules interfere with transduction pathway of A2AR, B7-H3, B7-H4, BTLA, CTLA-4, CD277, IDO, KIR, PD-1, LAG-3, TIM-3 or VISTA.

In a particular embodiment, small organic molecules interfere with transduction pathway of PD-1 and Tim-3. For example, they can interfere with molecules, receptors or enzymes involved in PD-1 and Tim-3 pathway.

In a particular embodiment, the small organic molecules interfere with Indoleamine-pyrrole 2,3-dioxygenase (IDO) inhibitor. IDO is involved in the tryptophan catabolism (Liu et al 2010, Vacchelli et al 2014, Zhai et al 2015). Examples of IDO inhibitors are described in WO 2014150677. Examples of IDO inhibitors include without limitation 1-methyl-tryptophan (IMT), β-(3-benzofuranyl)-alanine, β-(3-benzo(b)thienyl)-alanine), 6-nitro-tryptophan, 6-fluoro-tryptophan, 4-methyl-tryptophan, 5-methyl tryptophan, 6-methyl-tryptophan, 5-methoxy-tryptophan, 5-hydroxy-tryptophan, indole 3-carbinol, 3,3′-diindolylmethane, epigallocatechin gallate, 5-Br-4-Cl-indoxyl 1,3-diacetate, 9-vinylcarbazole, acemetacin, 5-bromo-tryptophan, 5-bromoindoxyl diacetate, 3-Amino-naphtoic acid, pyrrolidine dithiocarbamate, 4-phenylimidazole a brassinin derivative, a thiohydantoin derivative, a p-carboline derivative or a brassilexin derivative. In a particular embodiment, the IDO inhibitor is selected from 1-methyl-tryptophan, β-(3-benzofuranyl)-alanine, 6-nitro-L-tryptophan, 3-Amino-naphtoic acid and β-[3-benzo(b)thienyl]-alanine or a derivative or prodrug thereof.

In a particular embodiment, the inhibitor of IDO is Epacadostat, (INCB24360, INCB024360) has the following chemical formula in the art and refers to —N-(3-bromo-4-fluorophenyl)-N′-hydroxy-4-{[2-(sulfamoylamino)-6thyl]amino}-1,2,5-oxadiazole-3 carboximidamide:

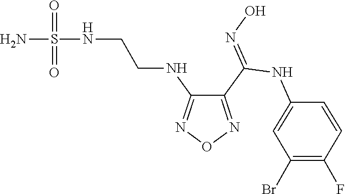 [SMILES: NS(=O)(=O)NCCNc1nonc1/C(=N/O)Nc1ccc(F)c(Br)c1]

In a particular embodiment, the inhibitor is BGB324, also called R428, such as described in WO2009054864, refers to 1H-1,2,4-Triazole-3,5-diamine, 1-(6,7-dihydro-5H-benzo[6,7]cyclohepta[1,2-c]pyridazin-3-yl)-N3-[(7S)-6,7,8,9-tetrahydro-7-(1-pyrrolidinyl)-5H-benzocyclohepten-2-yl]- and has the following formula in the art:

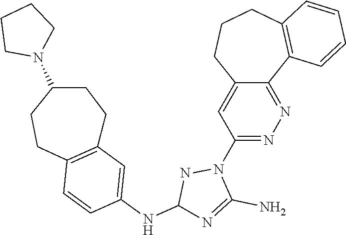

In a particular embodiment, the inhibitor is CA-170 (or AUPM-170): an oral, small molecule immune checkpoint antagonist targeting programmed death ligand-1 (PD-L1) and V-domain Ig suppressor of T cell activation (VISTA) (Liu et al 2015). Preclinical data of CA-170 are presented by Curis Collaborator and Aurigene on November at ACR-NCI-EORTC International Conference on Molecular Targets and Cancer Therapeutics.

In some embodiments, the immune checkpoint inhibitor is an aptamer.

Typically, the aptamers are directed against A2AR, B7-H3, B7-H4, BTLA, CTLA-4, CD277, IDO, KIR, PD-1, LAG-3, TIM-3 or VISTA.

In a particular embodiment, aptamers are DNA aptamers such as described in Prodeus et al 2015. A major disadvantage of aptamers as therapeutic entities is their poor pharmacokinetic profiles, as these short DNA strands are rapidly removed from circulation due to renal filtration. Thus, aptamers according to the invention are conjugated to with high molecular weight polymers such as polyethylene glycol (PEG). In a particular embodiment, the aptamer is an anti-PD-1 aptamer. Particularly, the anti-PD-1 aptamer is MP7 pegylated as described in Prodeus et al 2015.

The population of oncogenic cells modified and/or the vaccine composition according the invention is also provided in a kit.

Accordingly, in a fourth aspect, the invention relates to a kit comprising the population of oncogenic modified according to the invention and/or vaccine composition according to the invention for use in the treatment of a cancer and/or resistant cancer.

Thus, the invention relates to a kit comprising a population of oncogenic modified and/or vaccine composition according to claim 7 for use in the treatment of a cancer and/or resistant cancer, wherein the population of oncogenic modified is obtained according to the following steps:
i. obtaining a population of oncogenic cells from a subject suffering from a cancer; and
ii. treating said cells with a fusion protein comprising an AAC-11 leucine-zipper (LZ) derived peptide which is fused to at least one heterologous polypeptide

In a particular embodiment, the invention relates to a kit comprising vaccine composition and/or the combined preparation according to the invention and an information leaflet providing instructions for immunization. The kit comprises also the all materials for the administration of the products.

In a particular embodiment, the kit according to the invention comprises further other suitable adjuvants as described above or excipients. In a particular embodiment, the adjuvant is the fusion protein as described above. Typically, the adjuvant may be used alone, or two or more may be used in combination.

In particular, if there is a synergy effect among the adjuvants, it is preferable that two or more adjuvants exerting the synergy effect are used in combination. In other cases, an adjuvant may be used alone, but in accordance with the purpose, the adjuvant may be used in combination.

The invention will be further illustrated by the following figures and examples. However, these examples and figures should not be interpreted in any way as limiting the scope of the present invention.

### FIGURES

FIG. 1: RT53 treatment increases APL mice survival. (A) The indicated cells were left untreated or exposed to increasing concentrations of RT53 for 20 h. Cell death induced by peptide treatment was measured by lactate dehydrogenase (LDH) release. Data are means±s.e.m. (n=3). (B) APL spleen cells were exposed to 5 μM of RT53 in the presence or absence of 50 μM zVAD-fmk or 50 μM Necrostatin-1 (Nec-1) for 3 h. Necrotic cell death was monitored by lactate dehydrogenase (LDH) release from cells into the culture medium. The obtained values were normalized to those of the maximum LDH released (completely lysed) control. Data are means±s.e.m. (n=3). (C) 104 APL blasts were inoculated intravenously (i.v.) into FVB/N mice at day zero. Mice were then either left untreated (n=6), treated with ATRA (5 mg, subcutaneous implantation of 21-day release pellets, n=6) at day 6 or injected intraperitoneally (i.p.) with RT53 (2.4 mg/kg in normal saline) at day 10 every day for a total of seven doses (n=6). Survival curves were analyzed with the Mantel-Cox test. (D) APL mice obtained as in (D) were either left untreated (n=6), injected i.p. with RT53 (2.4 mg/kg in normal saline) at day 10 every other day for a total of seven doses (D10 Q2D schedule, n=6) or at day every day for a total of seven doses (D20 schedule, n=4). Survival curves were analyzed with the Mantel-Cox test.

FIG. 2: Inhibition of APL progression by prophylactic vaccination with RT53-treated APL blasts. (A) APL blasts in basal RPMI medium were left untreated or treated with either 2.5 μM of RT53 for 6 h (CRT exposure analysis) or 10 μM of RT53 for 1 h (HMGB1 and ATP release analysis). Extracellular HMGB1 (left) and ATP (middle) were then measured in the culture supernatant by ELISA and ATP-bioluminescence assays, respectively, and surface exposure of CRT (right) detected by FACS analysis. (B) APL blasts were exposed to 30 μM RT53 in basal RPMI medium for 3 h for cell death induction and the whole suspension was injected subcutaneously (2×106 cells) into the left flanks of FVB/N mice. Twelve days later, the vaccinated (n=8) or control mice (n=7) were injected i.v. with live 104 APL blasts. Survival curves were analyzed with the Mantel-Cox test. The schematic protocol used is illustrated (right).

FIG. 3: Tumor specificity and long-lasting effect of prophylactic vaccination with RT53-treated APL blasts. (A) FVB/N mice were vaccinated with RT53-treated APL blasts (n=10), RT53-treated spleen cells from healthy mice (n=5), or the indicated RT53-treated cells (n=5 per group) using the same protocol as in FIG. 2B. Twelve days later, the vaccinated or control mice were injected i.v. with live 104 APL blasts. Survival curves were analyzed with the Mantel-Cox test. (B) Surviving mice from FIG. 2B were challenged with 104 live APL spleen blasts 107 days (group 1, n=5) or 226 days (group 2, n=4) after initial APL engraftment. Survival curves were analyzed with the Mantel-Cox test. The schematic protocol used is illustrated (lower panel).

FIG. 4. Requirement of CD4+ and CD8+ T cells for prolonged survival induced by vaccination with RT53-treated APL blasts. FVB/N mice were depleted of either CD4+, CD8+ or both T cell populations by bi-weekly i.p. injection of 0.2 mg of T cell-type specific monoclonal antibodies starting 2 weeks before experiments. Injections were then performed 2 times per week during the study period. The efficacy of depletion was monitored by flow cytometric analysis (right panel). Depleted (n=5 per group) or naive mice (n=10) were then vaccinated with RT53-treated APL blasts and injected i.v. with live 104 APL blasts (left, upper panel). Survival curves were analyzed with the Mantel-Cox test. The schematic protocol used is illustrated (left, lower panel).

FIG. 5: Therapeutic efficacy of RT53-treated APL blasts vaccination in well-established leukemia. 104 APL blasts were inoculated i.v. into FVB/N mice at day zero. Mice were then vaccinated with RT53-treated APL blasts 3 or 10 days after leukemia engraftment (n=5 per group). Survival curves were analyzed with the Mantel-Cox test. The schematic protocol used is illustrated (right panel).

FIG. 6: RT39 triggers calreticulin exposure as well as the release of HMGB1 and ATP. ((A) U2OS cells were left untreated or exposed to increasing concentrations of RT39 for 3 h. Extracellular HMGB1 was then measured in the culture supernatant by an ELISA assay. Data are means±s.e.m. (n=3). (B) U2OS cells were left untreated or exposed to increasing concentrations of RT39 for 3 h. Extracellular ATP was then measured in the culture supernatant using an ATP-bioluminescence assay. Data are means±s.e.m. (n=3).

FIG. 7: Inhibition of APL progression by prophylactic vaccination with RT39-treated APL blasts. APL blasts were exposed to 30 μM RT39 in basal RPMI medium for 3 h for cell death induction and the whole suspension was injected subcutaneously (2×10⁶ cells) into the left flanks of FVB/N mice. Twelve days later, the vaccinated (n=8) or control mice (n=8) were injected i.v. with live 10⁴ APL blasts. Survival curves were analyzed with the Mantel-Cox test. The schematic protocol used is illustrated (right).

### EXAMPLE

Material & Methods

Peptides

Peptides were synthesized by Proteogenix (Strasbourg, France) and were >95% pure as verified by HPLC and mass spectrographic analysis.

Peptides sequence of RT53 and RT39 are the following:

RT53:
SEQ ID NO: 18
RQIKIWFQNRRMKWKKAKLNAEKLKDFKIRLQYFARGLQVYIRQLRLAL
QGKT
RT39:
SEQ ID NO: 19
RQIKIWFQNRRMKWKKLQYFARGLQVYIRQLRLALQGKT

The penetratin sequence is underlined.

Cell Lines and Chemicals

NB4 (purchased from ATCC), UF-1 (provided by Dr. Y. Ikeda, Tokyo, Japan), HUT-78 (provided by Dr. A. Marie-Cardine, INSERM U976, Paris, France) and B16F10 (provided by Dr. M. Dutreix, CNRS UMR3347, INSERM 1021, Paris, France) were used for the experiments. Cells were grown in RPMI 1640 medium supplemented with 10% foetal calf serum, L-glutamine (2 mM), 1 mM Hepes and 200 ug/ml penicillin/streptomycin antibiotics (Gibco). All cells were maintained at 37° C. in humidified 5% C02 atmosphere. All chemicals were purchased from Sigma.

Lactate Dehydrogenase, ATP and HMGB1 Release Assays

Release of lactate dehydrogenase (LDH) and ATP in the culture medium were assessed with the CytoTox 96 Non-Radioactive Cytotoxicity Assay and Enliten ATP Assay, respectively (Promega, Madison, WI, USA). HMGB1 release in the culture medium was assessed with the HMGB1 ELISA kit (IBL International, Hamburg, Germany).

Electronic Microscopy

Samples were fixed in 3% glutaraldehyde in phosphate buffer, pH 7.4 for 1 hour, washed, post-fixed with 1% osmium tetroxide in 0.1 M phosphate buffer and then gradually dehydrated in 70, 90 and 100% ethanol. After 10 min in a 1:2 mixture of epoxy propane and epoxy resin and 10 min in epon, samples were embedded in epoxy resin and polymerized at 60° C. for 24 h. After polymerisation, ultrathin sections of 90 nm were cut with an ultra-microtome (Reichert ultracut S), stained with uranyl acetate and Reynold's lead and observed with a transmission electron microscope (JEOL 1011). Acquisition was performed with a Gatan Orius 1000 CCD camera.

Determination of Surface-Exposed CRT

CRT exposure was assessed by surface immunostaining and flow cytometry. In brief, APL blasts cells (106 cells per mL) in RPMI 1640 medium supplemented with 10% foetal calf serum, L-glutamine (2 mM), 1 mM Hepes and 200 ug/ml penicillin/streptomycin antibiotics plated in 24 well plate were treated overnight with increasing concentrations of RT53 or RT39 peptides. Cells were washed with PBS (Phosphate-Buffered Saline), harvested and plated in 96-well round bottomed microtiter plates and incubated in blocking solution for 45 min (Blocking Solution Image-iT® Fixation/Permebilization Kit Cat. #R37602, Thermofischer Scientific). After 1×wash with PBS, cells were stained with anti-calreticulin primary antibody (Calreticulin (D3E6) XP® Rabbit mAb #12238, Cell Signaling). Goat anti-rabbit Alexa Fluor 488 was used as secondary antibody after another PBS wash (Alexa Fluor® 488 goat anti-rabbit IgG secondary antibody Cat. #A11034, Thermofischer Scientific). Cells were then analyzed with a CytoFLEX Flow Cytometer (Beckman and Coulter).

Ethics Statement

This study has been carried out in accordance with the EC Directive 86/609/EEC for animal experiments and was approved by the Committee for Experimental Animal Studies of the University of Paris 7 Institute Board Ethics (Protocol Number: 2303.01). Animals were housed and bred at our animal facility (Institut de Recherche Saint-Louis, Saint Louis Hospital, Paris, France) in vented animal cabinets under controlled temperature (22° C.) and 12 h light-dark cycle under pathogen-free conditions and were allowed food and water ad libitum.

Preclinical Acute Promyelocytic Leukemia-Transplantable Mice Model

APL blasts (provided by Drs. M. Bishop and S. Kogan, UCSF, USA) origin from the spleen of mice bearing the human PML-RARA cDNA construct driven by a myeloid linage specific promoter (hMRP8) in the FVB/N inbred strain of mice. For amplification, cells (1×105 or 1×106) were suspended in PBS and transplanted by intravenous (i.v) tail injection (200 uL) into female syngeneic recipient mice (5-6 weeks old). Establishment of leukemia was assessed by a decrease in blood platelet counts approx. 3 weeks after graft. Spleen cells from a primary recipient were collected, washed, re-suspended in PBS and injected (104 cells/mouse; 200 uL) into the tail veins of male FVB/N mice (7-8 weeks old) for experiments. For direct treatment experiments, mice were treated daily or every other day (i.p) with normal saline, RT53 or RT39 at 2.4 mg/kg in normal saline starting from day 10 or day 20 for a total of 7 injections.

RT53 or RT39-Treated APL Blasts Vaccination Assay

2×106 live cells from primary recipients' spleens or the indicated cells were washed in PBS and resuspended in 200 μl of serum-free RPMI medium. The cells were then exposed to 30 μM RT53 for 3 h for cell death induction and the whole suspension of RT53 or RT39-treated cells was injected subcutaneously (2×106 cells) into the left flanks of FVB/N syngeneic mice. For leukemia induction, the mice were injected i.v. with 1×104 blast cells from primary recipients' spleens at the indicated time.

T Cell Depletion

Mice were depleted of either CD4+, CD8+ or both T cell populations by bi-weekly i.p. injection of 0.2 mg of ascites fluids containing an anti-CD4 or -CD8 antibody starting 2 weeks before experiments. Injections were then performed 2 times per week during the study period. Blood was collected by submandibular bleeding. PBMC were labelled with a mix of anti-CD3E-APC (MACS), anti-CD4-PE (MACS) and anti-CD8-APC-cy7 (BD Biosciences) (2.5 μl, 30 min, 4° C.). Red blood cells were then lysed in ACK buffer for 7 min at RT. The efficacy of depletion was monitored using Canto II (BD Biosciences) cytometer and data analyzed with FlowJo software.

Results

RT53 Possess Direct Antileukemic Properties

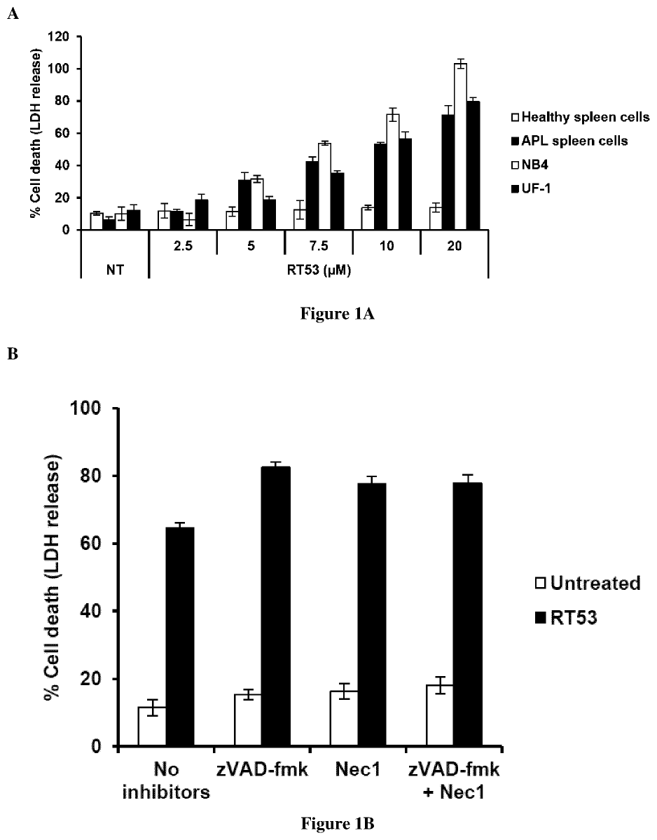

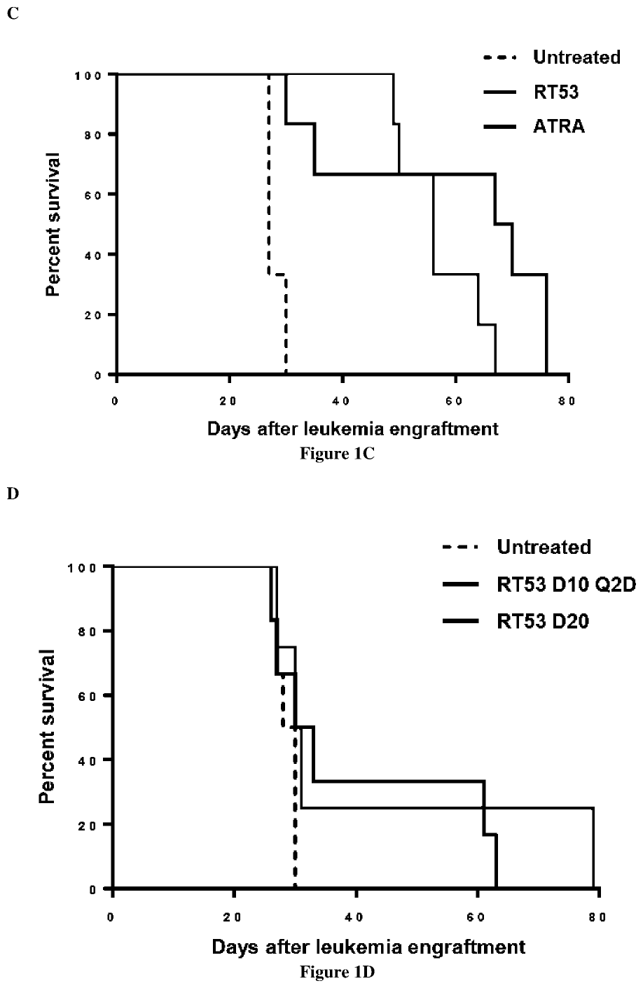

To test the antileukemic properties of RT53, we first exposed human all-trans retinoic acid (ATRA)-sensitive (NB4) and ATRA-resistant (UF-1) acute promyelocytic leukemia (APL) cells as well as mouse APL spleen blast cells derived from hMRP8-PML-RARA transgenic mice9 to increasing concentrations of RT53. As shown in FIG. 1A, RT53 decreased viability of all the tested cells through the rapid loss of plasma membrane integrity, as detected by the release of the intracellular enzyme lactate dehydrogenase (LDH). Importantly, no cell death was detected in spleen cells from healthy mice even at the highest concentration tested, indicating that RT53 exhibits selective cytotoxicity toward leukemic cells, but not normal cells. The observed LDH release upon RT53 treatment indicates that leukemic cells death is associated with loss of outer cell membrane integrity and cytoplasmic leakage, which are characteristic features of necrosis. In line with this hypothesis, ultrastructural analysis of APL spleen cells exposed to RT53 revealed an obvious necrotic morphology of the leukemic blasts, with loss of plasma membrane integrity and cytoplasmic swelling without morphological signs of nuclear apoptosis (data not shown). No changes in cellular integrity or ruptured cells could be detected in the normal spleen cells, confirming that RT53 exhibit high specificity towards leukemic cells (data not shown). Finally, the killing activity of RT53 was not hampered by inhibitors of apoptosis or necroptosis (FIG. 1B), confirming that, as previously observed in other settings^{6, 8,} RT53-induced leukemic blasts death occurs through a non-regulated form of necrosis.

To explore the in vivo therapeutic potential of RT53 as a treatment of acute leukemia, we used a well-characterized preclinical APL model bearing the human PML-RARA oncogene which mimics human APL, both in its characteristics and its response to conventional therapeutic drugs such as ATRA and arsenic trioxide^{9, 10}. In this model, 100% of the syngeneic mice (FVB/N) transplanted with 104 primary recipients' spleen blasts developed an APL and succumbed within 30 days (FIG. 1C). APL mice treated with RT53 (2.4 mg/kg) for 7 days starting on day 10 after leukemia engraftment had a significantly extended survival compared to control mice (P<0.0001). Survival of APL mice treated with RT53 was significantly (P<0.0001) superior to that of mice treated by ATRA (FIG. 1D). Similar survival advantage was obtained when RT53 was administered every other day for a total of seven administrations (D10 Q2D schedule, FIG. 1D). RT53 treatment starting on day 20 after leukemia engraftment, when leukemia is fully established¹¹ as shown by standardized minimal residual disease (MRD) monitoring (high level of PML-RARα transcripts in PBL, bone marrow and spleen), also prolonged the survival of leukemic mice (D20 schedule, FIG. 1D). To our knowledge, no therapeutic approach has demonstrated such a pronounced effect on survival of mice with comparable advanced disease stage in this preclinical model. No organ toxicity (macroscopic or microscopic) was noted with either treatment schedule (data not shown). Therefore, these results indicate that RT53 possesses robust antileukemic activity both in vitro and in vivo.

A Vaccine Comprising RT53-Treated APL Cells Induces Long-Term Survival

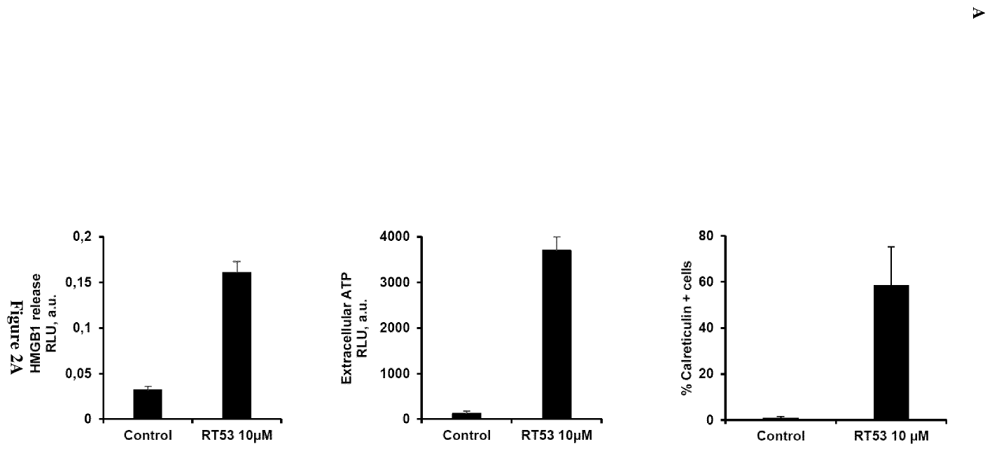

Anticancer chemotherapies are particularly effective when they induce immunogenic cell death (ICD), thus eliciting an antitumor immune response¹². We therefore investigated whether RT53 treatment of APL spleen cells would be able to induce the key known biomarkers of ICD, which include the endoplasmic reticulum (ER) chaperone calreticulin (CRT) surface exposure and the release of the chromatin protein high mobility group box1 protein (HMGB1) as well as ATP13. As showed in FIG. 2A, RT53 treatment triggered the release of both HMGB1 and ATP in the culture medium, detected by ELISA and ATP-bioluminescence assays, respectively, as well as surface exposure of CRT, detectable by FACS analysis, indicating that RT53 can induce all tested characteristics of ICD. Similar data were obtained with the human APL cells NB4 (not shown). To further investigate the capacity of RT53 to induce an antileukemic response, we took advantage of the APL preclinical model, which is based on immunocompetent FVB/N mice, to develop a prophylactic tumor vaccination model (FIG. 2B). Interestingly, 7 out of 8 mice vaccinated subcutaneously with RT53-exposed APL spleen blast cells did not develop disease after APL engraftment (FIG. 2B), indicating prophylactic effect of RT53-exposed APL cells. Eight months after APL engraftment, surviving animals were found disease-free by MRD monitoring. These data indicate that a simple vaccine constituted by RT53-treated APL cells triggered a very effective prophylaxis, protecting against the development of leukemia.

The Prophylactic Effect Generated by the RT53-Treated APL Cells Vaccine is Tumor Type Specific and Long-Lasting

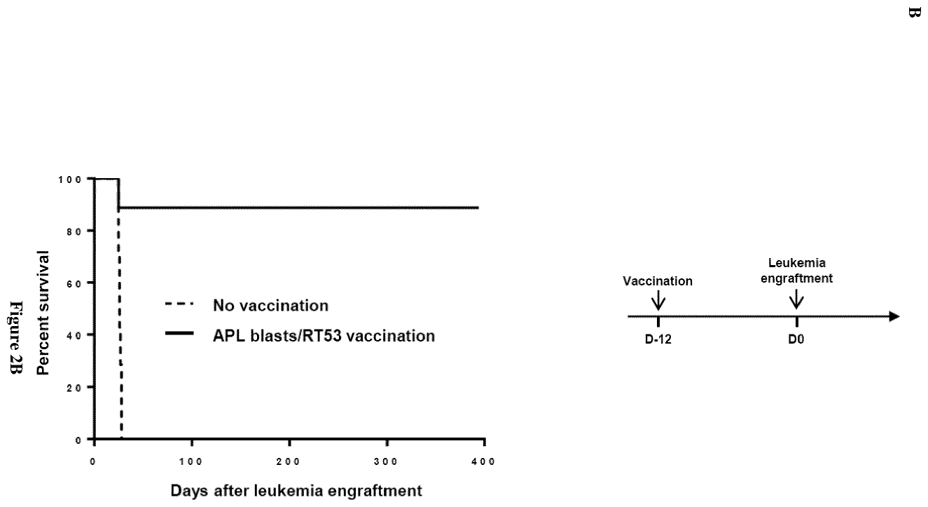

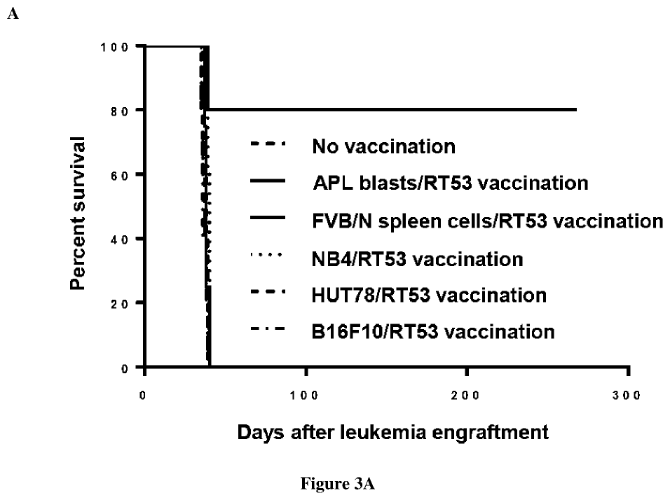

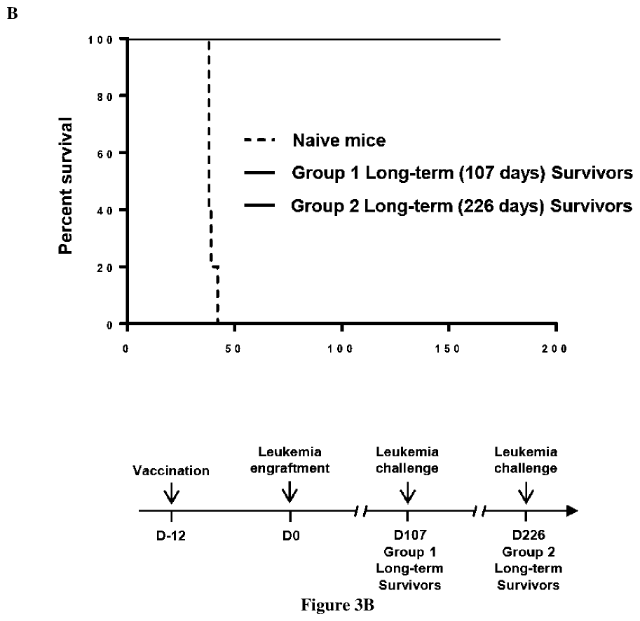

To demonstrate the specificity of RT53-exposed APL cells prophylactic effect, mice were vaccinated subcutaneously with various human (NB4, HUT78) or mouse (B16F10) cancerous cells treated with RT53. As shown in FIG. 3A, none of the tumor cells generated protection against leukemia development, indicating that the protection induced by RT53-treated APL cells is tumor specific. Moreover, the absence of protection observed following vaccination with RT53-treated NB4 cells suggests that immune clearance of leukemic cells in RT53-exposed APL cells vaccinated animals does not rely on the recognition of the unique PML-RARα fusion protein, which is also expressed in NB4 cells. Finally, vaccination with RT53-treated spleen cells from healthy FVB/N mice generated no protection (FIG. 3A), indicating that the prophylactic effect against APL was exclusively triggered by the RT53-treated leukemic APL cells.

We next determined whether surviving mice developed long-lasting antileukemic protection. At 107 or 226 days after initial leukemia engraftment, survivors from FIG. 2B or control mice were challenged with 104 live APL spleen blast cells, in the absence of any further therapy. Strikingly, all vaccinated animals that received vaccination were protected from APL cells challenge, whereas all control mice succumbed to leukemia within 40 days (FIG. 3B). These results indicate that single vaccination with RT53-exposed APL cells induces eradication of a rapidly fatal tumor burden and evokes effective, long-lasting prophylaxis capable of preventing leukemia. Moreover, when we inoculated 107 spleen cells originating from long-term survivors vaccinated with RT53-treated APL blasts into secondary recipients, none of the injected mice developed APL (followed up >200 days; not shown), suggesting that this vaccine leads to eradication of APL-initiating cells. Injection of 104 spleen cells from unvaccinated APL mice was sufficient to establish APL and all recipients died (not shown).

CD4+ T Cells are Critical for the Induction of Prolonged Survival Induced by the RT53-Treated APL Cells Vaccine

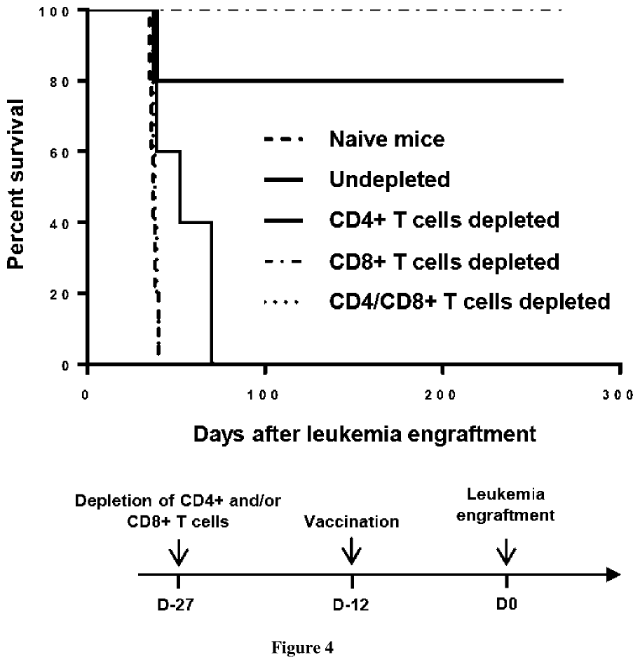

To determine the cells involved in the prophylactic effect of RT53-treated APL cells vaccination, mice were depleted of CD4+ T, CD8+ T or both T cell populations using cell type-specific antibodies. Depletion of CD4+ T cells notably reduced vaccine-induced protection, whereas depletion of CD8+ T cells had no effect on vaccine efficacy (FIG. 4), demonstrating the essential role of CD4+ T cells in the induction of effective antileukemic immunity. As CD4+ T cells are crucial in the establishment of immune memory¹⁴, our results might explain the protective effect observed in FIG. 3B. However, complete loss of protection was observed in mice that were depleted of both T-cell populations (FIG. 4), indicating that the antileukemic response generated by RT53-treated APL cells vaccination required the presence of both CD4+ and CD8+ T cells. Although the precise mechanisms involved in vaccination-induced protection remain to be defined, the observation that leukemia development is effectively contained in mice depleted for CD8+ T cells is suggestive of the induction of innate immune cells, such as macrophages and natural killer cells, which can be activated by CD4+ T cells¹⁵. A cytotoxic activity of CD4+ T cells toward the leukemic cells is also possible, as witnessed in different experimental tumor settings¹⁶¹⁸. Of note, depletion of either CD4+, CD8+ or both T cell populations in long-term survivors from FIG. 2B did not result in APL (not shown), indicating that CD4+ and/or CD8+ T cells were not necessary for the maintenance of the antileukemic effects of RT53-treated APL cells vaccination and suggesting strongly that this vaccination leads to a cure of the mice.

The RT53-Treated APL Cells Vaccine is Effective in Mice with Well-Established Leukemia

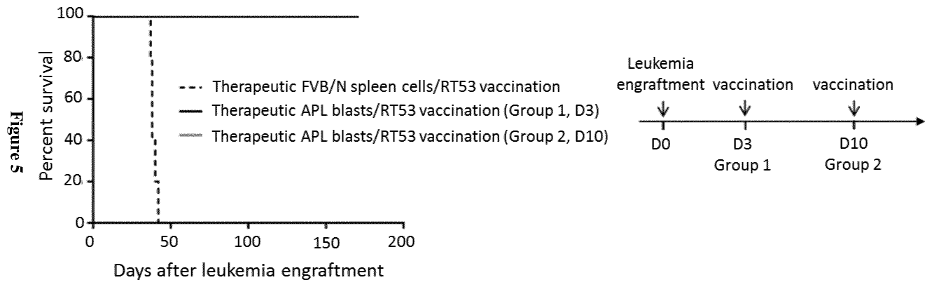

Having shown that RT53-treated APL cells vaccination can elicit an efficient prophylactic antileukemic effect, we next tested the therapeutic benefit of the vaccine in mice with well-established leukemia. For that purpose, mice received the RT53-treated APL cells 3 (rising disease) or 10 (well-established disease) 11 days after APL cell engraftment and the onset of the disease were compared to that of non-vaccinated controls. Very interestingly, 100% of the vaccinated mice were protected from leukemia development and remained disease-free through 80 days of observation, irrespective of the immunization schedule (FIG. 5, lower panel). These data indicate that therapeutic administration of the RT53-treated APL cells vaccine resulted in eradication of leukemic cells in all the tested mice, even when vaccination was delayed until 10 days after tumor inoculation, indicating the effectiveness of this approach.

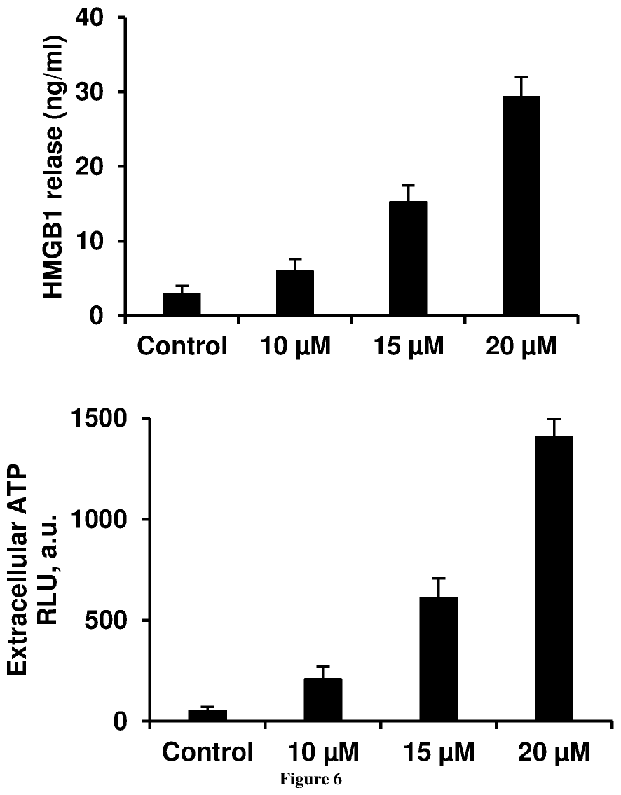

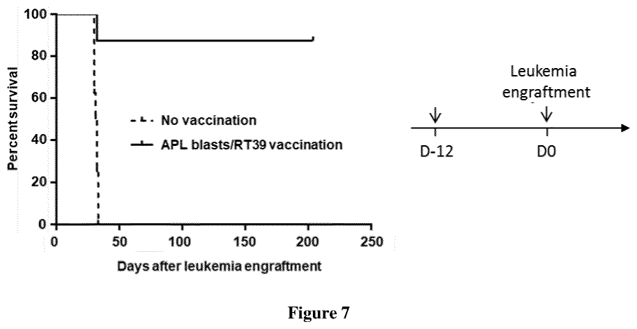

Inventors have also demonstrated an inhibition effect of APL progression by a prophylactic vaccination with RT39-treated APL blasts (FIGS. 6 & 7).

Conclusion

By using a well-established, aggressive APL model, inventors showed that single vaccination with a vaccine comprising whole leukemic cells exposed to a fusion peptide comprising an AAC-11 leucine-zipper (LZ) derived peptide with penetratin (such as RT53 or RT39), shown here to induce immunogenic cell death, protected against the development of leukemia in vivo both prophylactically and therapeutically. Cure of the tumor was observed and the vaccinated animals were protected against subsequent leukemia challenge in the absence of any further boosts, through the generation of long lasting, protective tumor specific responses involving CD4+ T cells. Indeed, the use of whole tumor cells lysates, obtained after in vitro treatment of cancerous cells with the peptides of the invention allows, upon injection, to reach sufficient antigen concentration in dendritic cells (DCs) to allow their activation. DCs cells are known to drive both CD4+ and CD8+ T cell responses. CD4+ T cells are needed for optimal and sustained effector CD8+ T cell responses as well as induction and maintenance of CD8+ memory²⁶⁻²⁷. The inventors clearly show that the anticancer response generated by peptide-treated APL cells vaccination required the presence of both CD4+ and CD8+ T cells, indicating activation of both T cell populations. One explication for this activation is that peptide-induced whole cell lysates, as tumor lysates, should contain all relevant major histocompatibility complex class I and class II epitopes capable of stimulating CD8+ and CD4+ T cells, respectively²⁸. In addition, whole cell lysates such as the ones obtained with the peptide of the invention could greatly diminish the chance of tumor escape compared to using single epitope vaccines. Furthermore, the use of whole tumor cells eliminates the need to define, test and select for immunodominant epitopes. The tumor cells could be autologous, i.e. obtained from the patients, or allogeneic “off-the-shelf”. Tumor cells from each patient potentially carry gene mutations encoding for unique tumor associated antigens (TAAs) that are important in stimulating effective and long-lasting anti-tumor responses in the patient.

Such vaccine-based approach is practical, as it does not require the knowledge of specific tumor antigens, is not limited by the HLA phenotype and is safe because the antitumor effect is obtained without the use of potentially toxic immunostimulatory adjuvants in the vaccine. Indeed in view of the overwhelming importance of activated DCs in the initiation of therapeutic CD4+ and CD8+ T cell responses, therapeutic cancer vaccines must activate DCs with adjuvants in order to increase the immunogenicity of whole-cell tumor vaccines. Appropriate adjuvants include haptens, TLR (toll-like receptor) or CD40 agonists, cytokines, or activators of IFN genes²⁵. Although adjuvants show promising results to favor therapeutic vaccines efficacy, at this time, only few immunostimulants have been approved for human use. Herein, surprisingly, the inventors do not use conventional adjuvants for the vaccine preparation: the lytic peptides used to prepare the herein described whole cell vaccines act as adjuvant, as they are still present when the vaccines are injected. As no toxicity nor adverse side effects were detected upon vaccination, these peptides could therefore be of great interest as innovative adjuvants for human clinic.

Because leukemic blasts can be easily obtained from the blood or bone marrow of patients at diagnosis, yielding sufficient material for clinical use, fusion peptide comprising an AAC-11 leucine-zipper (LZ) derived peptide with penetratin (such as RT53 or RT39) treated leukemic cells vaccines are a workable and effective strategy for immunotherapy of leukemia. Further, inventors demonstrated the single agent efficacy of a fusion peptide comprising an AAC-11 leucine-zipper (LZ) derived peptide with penetratin (such as RT53 or RT39) for the treatment of established leukemia, even at late stages, suggesting that a fusion peptide comprising an AAC-11 leucine-zipper (LZ) derived peptide with penetratin (such as RT53 or RT39) constitutes a therapeutic solution for patients who experienced multiple relapse because of resistance to approved therapies.

### REFERENCES

Throughout this application, various references describe the state of the art to which this invention pertains. The disclosures of these references are hereby incorporated by reference into the present disclosure.
1. Ellis L M, Hicklin D J. Resistance to Targeted Therapies: Refining Anticancer Therapy in the Era of Molecular Oncology. Clin Cancer Res 2009; 15:7471-8.
2. Neel D S, Bivona T G. Resistance is futile: overcoming resistance to targeted therapies in lung adenocarcinoma. NPJ Precis Oncol 2017; 1.
3. Martin S D, Coukos G, Holt R A, Nelson B H. Targeting the undruggable: immunotherapy meets personalized oncology in the genomic era. Annals of oncology: official journal of the European Society for Medical Oncology/ESMO 2015; 26:2367-74.
4. Thallinger C, Fureder T, Preusser M, Heller G, Mullauer L, Holler C, Prosch H, Frank N, Swierzewski R, Berger W, et al. Review of cancer treatment with immune checkpoint inhibitors: Current concepts, expectations, limitations and pitfalls. Wien Klin Wochenschr 2018; 130:85-91.
5. De Kouchkovsky I, Abdul-Hay M. ‘Acute myeloid leukemia: a comprehensive review and 2016 update’. Blood Cancer J 2016; 6:e441.
6. Jagot-Lacoussiere L, Kotula E, Villoutreix B O, Bruzzoni-Giovanelli H, Poyet J L. A Cell-Penetrating Peptide Targeting AAC-11 Specifically Induces Cancer Cells Death. Cancer research 2016; 76:5479-90.
7. Rigou P, Piddubnyak V, Faye A, Rain J C, Michel L, Calvo F, Poyet J L. The antiapoptotic protein AAC-11 interacts with and regulates Acinus-mediated DNA fragmentation. The EMBO journal 2009; 28:1576-88.
8. Pasquereau-Kotula E, Habault J, Kroemer G, Poyet J L. The anticancer peptide RT53 induces immunogenic cell death. PloS one 2018; 13:e0201220.
9. Brown D, Kogan S, Lagasse E, Weissman I, Alcalay M, Pelicci P G, Atwater S, Bishop J M. A PMLRARalpha transgene initiates murine acute promyelocytic leukemia. Proceedings of the National Academy of Sciences of the United States of America 1997; 94:2551-6.
10. Lallemand-Breitenbach V, Guillemin M C, Janin A, Daniel M T, Degos L, Kogan S C, Bishop J M, de The H. Retinoic acid and arsenic synergize to eradicate leukemic cells in a mouse model of acute promyelocytic leukemia. The Journal of experimental medicine 1999; 189:1043-52.
11. Pokorna K, Le Pogam C, Chopin M, Balitrand N, Reboul M, Cassinat B, Chomienne C, Padua R A, Pla M. Tracking the extramedullary PML-RARalpha-positive cell reservoirs in a preclinical model: biomarker of long-term drug efficacy. Mol Cell Probes 2013; 27:1-5.
12. Casares N, Pequignot M O, Tesniere A, Ghiringhelli F, Roux S, Chaput N, Schmitt E, Hamai A, Hervas-Stubbs S, Obeid M, et al. Caspase-dependent immunogenicity of doxorubicin-induced tumor cell death. The Journal of experimental medicine 2005; 202:1691-701.
13. Kepp O, Senovilla L, Vitale I, Vacchelli E, Adjemian S, Agostinis P, Apetoh L, Aranda F, Barnaba V, Bloy N, et al. Consensus guidelines for the detection of immunogenic cell death. Oncoimmunology 2014; 3:e955691.
14. Chen S A, Tsai M H, Wu F T, Hsiang A, Chen Y L, Lei H Y, Tzai T S, Leung H W, Jin Y T, Hsieh C L, et al. Induction of antitumor immunity with combination of HER2/neu DNA vaccine and interleukin 2 gene-modified tumor vaccine. Clin Cancer Res 2000; 6:4381-8.
15. Luckheeram R V, Zhou R, Verma A D, Xia B. CD4(+)T cells: differentiation and functions. Clin Dev Immunol 2012; 2012:925135.
16. Quezada S A, Simpson T R, Peggs K S, Merghoub T, Vider J, Fan X, Blasberg R, Yagita H, Muranski P, Antony P A, et al. Tumor-reactive CD4(+) T cells develop cytotoxic activity and eradicate large established melanoma after transfer into lymphopenic hosts. The Journal of experimental medicine 2010; 207:637-50.
17. Fu J, Zhang Z, Zhou L, Qi Z, Xing S, Lv J, Shi J, Fu B, Liu Z, Zhang J Y, et al. Impairment of CD4+ cytotoxic T cells predicts poor survival and high recurrence rates in patients with hepatocellular carcinoma. Hepatology 2013; 58:139-49.
18. Sharma R K, Yolcu E S, Srivastava A K, Shirwan H. CD4+ T cells play a critical role in the generation of primary and memory antitumor immune responses elicited by SA-4-1BBL and TAA-based vaccines in mouse tumor models. PloS one 2013; 8:e73145.
19. Tewari et al. AAC-11, a novel cDNA that inhibits apoptosis after growth factor withdrawal. Cancer Res. 1997 Sep. 15; 57(18):4063-9.
20. Morris et al. Functional identification of Api5 as a suppressor of E2F-dependent apoptosis in vivo. PLoS Genet. 2006 Nov. 17; 2(11):e196
21. Van den Berghe et al. FIF [fibroblast growth factor-2 (FGF-2)-interacting-factor], a nuclear putatively antiapoptotic factor, interacts specifically with FGF-2. Mol Endocrinol. 2000 November; 14(11):1709-24.
22. Kim et al. AAC-11 overexpression induces invasion and protects cervical cancer cells from apoptosis. Lab Invest. 2000 April; 80(4):587-94
23. Kim et al. Prevention of hepatocellular carcinoma in patients with chronic hepatitis B virus infection. Oncology. 2011; 81 Suppl 1:41-9.
24. Roden B. S et al. Opportunities and challenges for human papillomavirus vaccination in cancer. Nat Rev Cancer. 2018 April; 18(4):240-254.
25. Melief C. J. M et al. Therapeutic cancer vaccines. J Clin Invest. 2015 September; 125(9):3401-12.
26. Schoenberger S. P et al. T-cell help for cytotoxic T lymphocytes is mediated by CD40-CD40L interactions. Nature. 1998 Jun. 4; 393(6684):480-3.
27. Janssen E. M et al. CD4+ T cells are required for secondary expansion and memory in CD8+ T lymphocytes. Nature. 2003 Feb. 20; 421(6925):852-6.
28. Toes R. E et al. CD4 T cells and their role in antitumor immune responses. J Exp Med. 1999 Mar. 1; 189(5):753-6

1. A method for treating a cancer in a subject in need thereof comprising the following step:
i. obtaining a population of oncogenic cells from a subject suffering from a cancer;
ii. treating said oncogenic cells with a fusion protein comprising an AAC-11 leucine-zipper (LZ) derived peptide which is fused to at least one heterologous polypeptide; and
iii. administering to the subject a therapeutically effective amount of the population of the oncogenic cells modified in step ii).

2. The method for treating according to claim 1, wherein said population of oncogenic cells is obtained from blood, bone marrow or biopsy.

3. The method for treating according to claim 1, wherein said fusion protein comprises and/or consists of a sequence selected from the group consisting of SEQ ID NO:4, SEQ ID NO:6; SEQ ID NO:7, SEQ ID NO:8, SEQ ID NO:9, SEQ ID NO:10; SEQ ID NO:11; SEQ ID NO:13; and SEQ ID NO:14; SEQ ID NO:16; SEQ ID NO:17; SEQ ID NO:18; and SEQ ID NO:19.

4. The method for treating according to claim 1, wherein said fusion protein comprises and/or consists of a sequence SEQ ID NO: 18.

5. The method for treating according to claim 1, wherein said fusion protein comprises and/or consists, of a sequence SEQ ID NO: 19.

6. A vaccine composition comprising a population of oncogenic modified cells obtained according to the following steps:
a. obtaining a population of oncogenic cells from a subject suffering from a cancer; and
b. treating said cells with a fusion protein comprising an AAC-11 leucine-zipper (LZ) derived peptide which is fused to at least one heterologous polypeptide.

7. The vaccine composition according to claim 6 for use in the treatment of a cancer in a subject in need thereof.

8. The vaccine composition according to claim 6, wherein the cancer is selected from the following group but is not limited to: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous; adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; and roblastoma, malignant; Sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malig melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

9. The vaccine composition according to claim 8 wherein the cancer is resistant.

10. The method for treating according to claim 1, wherein the population of oncogenic cells modified is combined with a classical treatment.

11. A kit comprising the vaccine composition according to claim 6 for use in the treatment of a cancer and/or resistant cancer, wherein the population of oncogenic modified cells is obtained according to the following steps:
a. obtaining a population of oncogenic cells from a subject suffering from a cancer; and
b. treating said cells with a fusion protein comprising an AAC-11 leucine-zipper (LZ) derived peptide which is fused to at least one heterologous polypeptide.

12. The method for treating according to claim 1, wherein the cancer is selected from the following group but is not limited to: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous; adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; and roblastoma, malignant; Sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malig melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

13. The method for treating according to claim 12 wherein the cancer is resistant.
